SEQUENCE LISTING (62 sequences)

SEQ ID NO: 1 - protein, 23 aa, Artificial Sequence
  Synthetic
LKPFERDISTEIYQAGSTPSNGV

SEQ ID NO: 2 - protein, 27 aa, Artificial Sequence
  Synthetic
CGLKPFERDISTEIYQAGSTPSNGVGC

SEQ ID NO: 3 - protein, 24 aa, Artificial Sequence
  Synthetic
YNSASFSTFKSYGVSPTKLNDLSF

SEQ ID NO: 4 - protein, 28 aa, Artificial Sequence
  Synthetic
CGYNSASFSTFKSYGVSPTKLNDLSFGC

SEQ ID NO: 5 - protein, 14 aa, Artificial Sequence
  Synthetic
YNYKLPDDFTGSVI

SEQ ID NO: 6 - protein, 18 aa, Artificial Sequence
  Synthetic
CGYNYKLPDDFTGSVIGC

SEQ ID NO: 7 - protein, 20 aa, Artificial Sequence
  Synthetic
NSNNLDSKVGGNYNYLYRLF

SEQ ID NO: 8 - protein, 24 aa, Artificial Sequence
  Synthetic
CGNSNNLDSKVGGNYNYLYRLFGC

SEQ ID NO: 9 - protein, 21 aa, Artificial Sequence
  Synthetic
YQAGSTPSNGVEGFNSYFPLQ

SEQ ID NO: 10 - protein, 25 aa, Artificial Sequence
  Synthetic
CGYQAGSTPSNGVEGFNSYFPLQGC

SEQ ID NO: 11 - protein, 21 aa, Artificial Sequence
  Synthetic
FNSYFPLQSYGFQPTNGVGYQ

SEQ ID NO: 12 - protein, 25 aa, Artificial Sequence
  Synthetic
CGFNSYFPLQSYGFQPTNGVGYQGC

SEQ ID NO: 13 - protein, 21 aa, Artificial Sequence
  Synthetic
VLSFELLHAPATVSGPKKSTN

SEQ ID NO: 14 - protein, 25 aa, Artificial Sequence
  Synthetic
CGVLSFELLHAPATVSGPKKSTNGC

SEQ ID NO: 15 - protein, 13 aa, Artificial Sequence
  Synthetic
TIDGSSGVVNPVL

SEQ ID NO: 16 - protein, 17 aa, Artificial Sequence
  Synthetic
CGTIDGSSGVVNPVLGC

SEQ ID NO: 17 - protein, 18 aa, Artificial Sequence
  Synthetic
PVLEPIYDEPTTTTSVPL

SEQ ID NO: 18 - protein, 22 aa, Artificial Sequence
  Synthetic
CGPVLEPIYDEPTTTTSVPLGC

SEQ ID NO: 19 - protein, 19 aa, Artificial Sequence
  Synthetic
VTLAILTALRLSAYSSNIV

SEQ ID NO: 20 - protein, 23 aa, Artificial Sequence
  Synthetic
CGVTLAILTALRLSAYSSNIVGC

SEQ ID NO: 21 - protein, 23 aa, Artificial Sequence
  Synthetic
ETNILLNVPLHGTILTRPLLESE

SEQ ID NO: 22 - protein, 27 aa, Artificial Sequence
  Synthetic
CGETNILLNVPLHGTILTRPLLESEGC

SEQ ID NO: 23 - protein, 15 aa, Artificial Sequence
  Synthetic
IVFITLSFTLKRKTE

SEQ ID NO: 24 - protein, 19 aa, Artificial Sequence
  Synthetic
CGIVFITLSFTLKRKTEGC

SEQ ID NO: 25 - protein, 11 aa, Artificial Sequence
  Synthetic
SLELQDHNETS

SEQ ID NO: 26 - protein, 15 aa, Artificial Sequence
  Synthetic
CGSLELQDHNETSGC

SEQ ID NO: 27 - protein, 20 aa, Artificial Sequence
  Synthetic
KDPNFKDQVILLNKHIDAYK

SEQ ID NO: 28 - protein, 24 aa, Artificial Sequence
  Synthetic
CGKDPNFKDQVILLNKHIDAYKGC

SEQ ID NO: 29 - protein, 20 aa, Artificial Sequence
  Synthetic
KDQVILLNKHIDAYKTFPPT

SEQ ID NO: 30 - protein, 24 aa, Artificial Sequence
  Synthetic
CGKDQVILLNKHIDAYKTFPPTGC

SEQ ID NO: 31 - protein, 21 aa, Artificial Sequence
  Synthetic
YQAGSTPSNGVKGFNSYFPLQ

SEQ ID NO: 32 - protein, 25 aa, Artificial Sequence
  Synthetic
CGYQAGSTPSNGVKGFNSYFPLQGC

SEQ ID NO: 33 - protein, 21 aa, Artificial
  Synthetic
YQAGSTPSNGVNGFNSYFPLQ

SEQ ID NO: 34 - protein, 25 aa, Artificial Sequence
  Synthetic
CGYQAGSTPSNGVNGFNSYFPLQGC

SEQ ID NO: 35 - protein, 21 aa, Artificial Sequence
  Synthetic
FNSYFPLQSYGFQPTYGVGYQ

SEQ ID NO: 36 - protein, 25 aa, Artificial Sequence
  Synthetic
CGFNSYFPLQSYGFQPTYGVGYQGC

SEQ ID NO: 37 - protein, 22 aa, Artificial Sequence
  Synthetic
ENLLLYIDINGNLHPDSATLVS

SEQ ID NO: 38 - protein, 26 aa, Artificial Sequence
  Synthetic
CGENLLLYIDINGNLHPDSATLVSGC

SEQ ID NO: 39 - protein, 19 aa, Artificial Sequence
  Synthetic
SDIDITFLKKDAPYIVGDV

SEQ ID NO: 40 - protein, 23 aa, Artificial Sequence
  Synthetic
CGSDIDITFLKKDAPYIVGDVGC

SEQ ID NO: 41 - protein, 23 aa, Artificial Sequence
  Synthetic
NPTIQKDVLESNVKTTEVVGDII

SEQ ID NO: 42 - protein, 27 aa, Artificial Sequence
  Synthetic
CGNPTIQKDVLESNVKTTEVVGDIIGC

SEQ ID NO: 43 - protein, 19 aa, Artificial Sequence
  Synthetic
ILKPANNSLKITEEVGHTD

SEQ ID NO: 44 - protein, 23 aa, Artificial Sequence
  Synthetic
CGILKPANNSLKITEEVGHTDGC

SEQ ID NO: 45 - protein, 27 aa, Artificial Sequence
  Synthetic
SLQNSVLKLKVDTANPKTPKYKFVRIQ

SEQ ID NO: 46 - protein, 31 aa, Artificial Sequence
  Synthetic
CGSLQNSVLKLKVDTANPKTPKYKFVRIQGC

SEQ ID NO: 47 - protein, 22 aa, Artificial Sequence
  Synthetic
EIVDTVSALVYDNKLKAHKDKS

SEQ ID NO: 48 - protein, 26 aa, Artificial Sequence
  Synthetic
CGEIVDTVSALVYDNKLKAHKDKSGC

SEQ ID NO: 49 - protein, 22 aa, Artificial Sequence
  Synthetic
VSALVYDNKLKAHKDKSAQSFK

SEQ ID NO: 50 - protein, 26 aa, Artificial Sequence
  Synthetic
CGVSALVYDNKLKAHKDKSAQSFKGC

SEQ ID NO: 51 - protein, 19 aa, Artificial Sequence
  Synthetic
GVSLFWNSNVDRYPANSIV

SEQ ID NO: 52 - protein, 19 aa, Artificial Sequence
  Synthetic
GVSLFWNSNVDRYPANSIV

SEQ ID NO: 53 - protein, 19 aa, Artificial
  Synthetic
NSIVSRFDTRVLSNLNLPG

SEQ ID NO: 54 - protein, 23 aa, Artificial Sequence
  Synthetic
CGNSIVSRFDTRVLSNLNLPGGC

SEQ ID NO: 55 - protein, 21 aa, Artificial Sequence
  Synthetic
SKSLTENKYSQLDEEQPLEID

SEQ ID NO: 56 - protein, 25 aa, Artificial Sequence
  Synthetic
CGSKSLTENKYSQLDEEQPLEIDGC

SEQ ID NO: 57 - protein, 14 aa, Artificial Sequence
  Synthetic
LTENKYSQLDEEQP

SEQ ID NO: 58 - protein, 18 aa, Artificial Sequence
  Synthetic
CGLTENKYSQLDEEQPGC

SEQ ID NO: 59 - protein, 27 aa, Artificial Sequence
  Synthetic
SLQSSTQHQPYVVDDPSPIHFYSKWYI

SEQ ID NO: 60 - protein, 31 aa, Artificial Sequence
  Synthetic
CGSLQSSTQHQPYVVDDPSPIHFYSKWYIGC

SEQ ID NO: 61 - protein, 18 aa, Artificial Sequence
  Synthetic
TQHQPYVVDDPSPIHFYS

SEQ ID NO: 62 - protein, 22 aa, Artificial Sequence
  Synthetic
CGTQHQPYVVDDPSPIHFYSGC

(19) United States
(12) Patent Application Publication
(10) Pub. No.: US 2022/0125913 A1
(43) Pub. Date: Apr. 28, 2022

(54) SYNTHETIC PEPTIDES, AMINO-ACID SEQUENCES, AND COMPOSITIONS USEFUL FOR SARS-CoV-2 DETECTION AND COVID-19 PREVENTION

(71) Applicants: Jose Manuel Lozano Moreno, Bogotá (CO); Luz Mary Salazar Pulido, Bogotá (CO)

(72) Inventors: Jose Manuel Lozano Moreno, Bogotá (CO); Luz Mary Salazar Pulido, Bogotá (CO)

(21) Appl. No.: 17/511,090

(22) Filed: Oct. 26, 2021

Related U.S. Application Data
(60) Provisional application No. 63/105,478, filed on Oct. 26, 2020.

(51) Int. Cl. A61K 39/215; C12Q 1/6888; C07K 14/165; A61P 31/14

(52) U.S. Cl. CPC A61K 39/215; C12Q 1/6888; C07K 2317/34; A61P 31/14; C07K 14/165

(57) ABSTRACT

The present invention provides designing and synthesizing monomer, polymer, or multimer and formulations of amino-acid chains and peptides from SARS-CoV-2 which are subjected to specific peptide-backbone modifications after being screened by a rational selection which has proven to be highly antigenic and immunogenic useful for human antibody detection and stimulation in higher vertebrates as vaccine components. The so obtained peptides have a common functional motif of formula I:

$${}_{2} NH \text{-} {AA}_{1} \text{-} {AA}_{2} \text{-} {AA}_{3} \text{-} {( AA )}_{n - 1} \text{-} {( AA )}_{n} \text{-} COX \qquad Formula I$$

where X represents either a —CO—NH₂ or a -COOH function, AA is any amino-acid, AA₁ is the N-terminus amino-acid residue of a peptide fragment and AAₙ represents the C-terminus residue of peptide chain from 4 to 30 residues included but are not limited to any peptide sequence are either the 20 genetically coded L-amino-acids or their D-enantiomers even those named non-natural amino-acids as well as peptide-bond isostere forms on specific sequence sites.

This application claims the priority benefit under 35 U.S.C. section 119 of U.S. Provisional Patent Application No. 63/105,478 entitled “Peptides, Amino-Acid Sequences And Compositions Useful For Sars-Cov-2 Detection And Covid-19 Prevention” filed Oct. 26, 2020, which is in its entirety herein incorporated by reference.

### FIELD OF THE INVENTION

The invention belongs to the field of native and modified peptides presented as monomer, polymer and multimer forms, rational methodologies for designing and synthesizing biologically active short, medium, and large size amino-acid sequences from the SARS-Cov-2 virus genome, known as the causative agent of the so named COVID-19 disease, as well as compositions and formulations containing specific targeted sequences. More particularly, this invention relates to designing site-directed polypeptides, chemical modifications, and formulations thereof in a variety of adjuvant systems, as either single or multiple sub-unit mixtures lacking both toxic and hemolytic activities that may be used as immuno-detection probes, as well as are basic components of synthetic vaccine candidates for COVID-19 prevention.

In the present invention, specific peptide sequences are used as immuno-detection agents for structural, non-structural, and accessory proteins of the SARS-CoV-2 virus and some of them as potent antibody inducers in mammalians.

### SUMMARY

The present invention provides amino-acid chains and peptides from SARS-CoV-2 which are subjected to specific modifications after being screened by a rational selection which have proven to be highly antigenic and immunogenic. The so obtained peptides have a common a functional motif of formulas Ia and Ib:

₂NH—AA₁—AA₂AA₃—(AA)ₙ₋₁—(AA)ₙ—CONH₂ Formula Ia

₂NH—AA₁—AA₂—AA₃—(AA)ₙ₋₁—(AA)ₙ—COOH Formula Ib

where AA is any amino-acid, AA₁ is the N-terminus amino-acid residue of a peptide fragment and AAₙ represents the C-terminus residue of the peptide chain having from 4 to 30 residues included but are not limited to any peptide sequence and are either the 20 genetically coded L-amino-acids or their D-enantiomers as well as those named non-natural amino-acids as well as, peptide-bond isostere forms on specific sequence sites. Isostere-bond modification sites are selected from given amino-acid pairs from SARS-CoV-2 selected sequences as single peptide chains herein named as monomer forms.

SARS-CoV-2 peptide selected sequences can be also modified to introduce post-translation elements such as phosphate, methyl, mono and oligo saccharides, small and medium size peptide chains among others. Modifications included but are not limited to Tyr (tyrosine), Gln (glutamine), Asn (asparagine), Ser (serine) and Thr (threonine). Besides, Lys (lysine) residues can be positioned on given peptide motifs to further anchoring other or similar oligo sequences as dendrimers or MAPs (multiple peptide antigens) presenting sequences. Among the realm of peptide modifications, those consisting of introducing solubility inducing motifs can be used among PEG (polyethylene glycol) or even those to be positioned as hydrophobic motifs such as fatty acid small, medium, and large size chains including but not limited to palmitic, lauric, stearic, and fumaric acids among all saturated and non-saturated carbon chains.

On the other hand, monomer native and modified SARS-CoV-2 peptide forms are synthesized simultaneously with others having the same amino-acid sequence but including extra amino-acid pairs on both C- and N-terminal peptide ends to serve for obtaining and present them as high molecular weight polymer forms thereof. The formula II represents the group of SARS-CoV-2 peptides synthesized for obtaining one type of those sequences as polymer forms:

₂NH—Cys—AA₂—AA₃.AA₄—(AA)ₙ₋₁—Cys—CONH₂ Formula IIa

₂NH—Cys—AA₂—AA₃.AA₄—(AA)ₙ₋₁—Cys—COOH Formula IIb

where the N-terminus AA₁ is a Cys (cysteine) residue, as well as the C-terminus (AA)ₙ is a Cys (cysteine) residue. Residues at positions —AA.₂, and —(AA)ₙ₋₁ can be selected from those 20 D- or L-amino-acids such as those side-chain charged polar residues or side-chain functionalized residues such as Gln or Asn, or non-polar residues, or non-natural residues, as well as alcohol, carboxylic acids, primary and secondary amino groups or thiol functionalized side-chains or other less complex residues such as Ala (alanine) or Gly (glycine) all considered to be used to introduce spacer motifs that can be located on a given SARS-Cov-2 selected amino-acid sequence.

Cysteine residues located at both C- and N-terminal residues serve for obtaining polymer peptides through disulfide-bond oxidation allowed upon being submitted to an oxygen-controlled stream at a pH adjusted between 6.5 to 7.5 under constant stirring at room temperature for a defined time-period from 1 to 24 hours.

All native and modified peptide sequences are aimed to function as SARS-CoV-2 immuno-detection, as well as for COVID-19 prevention tools and can be obtained after performing a rational designing which also included some bioinformatics besides knowledge on immunology, vaccinology, and molecular designing. Compositions and formulations thereof in a variety of adjuvant systems including those allowed to human use containing these SARS-CoV-2 peptide-epitopes, methods to obtain them as either monomer, multimer or polymer forms and uses thereof are also described.

The peptides and compositions thereof described herein, have clinical potential as immuno-detection tools useful for ELISA, immuno-chromatography, and western-blot-based assays as well as, for antibody stimulation in mammals when properly formulated as immunogens on a variety of veterinary and human adjuvant and delivery systems and further those have potential use for preventing the human COVID-19 disease.

### BACKGROUND OF THE INVENTION

A pandemic is an epidemic of disease that has spread across a large region, such as multiple continents, that affects a substantial number of people. There a several pandemics that have been documented throughout human history, such as smallpox and tuberculosis. The most fatal pandemic in recorded history was the Black Death (also known as The Plague), which killed an estimated 75-200 million people in the 14th century. Other notable pandemics include the 1918 influenza pandemic (Spanish flu) and the recent Covid-19 pandemic.

The basic strategies to control of an outbreak include containment and mitigation. Containment generally includes contact tracing and isolating infected individuals to stop the disease from spreading. Mitigation is employed when it is no longer possible to contain the spread of the disease. In this stage, additional measures are taken to try to slow the spread of the disease and mitigate its effects on society and the healthcare system.

Managing an infectious disease outbreak can include efforts to decrease the epidemic peak, known as “flattening the curve.” This helps decrease the risk of health services being overwhelmed and provides more time for the development of vaccines and/or treatments. Interventions may be taken to manage the outbreak including: personal preventive measures such as hand hygiene, wearing face-masks, and self-quarantine; community measures aimed at social distancing such as closing schools and cancelling mass gatherings; community engagement to encourage acceptance and participation in such interventions; and environmental measures such as cleaning of surfaces.

Another strategy, suppression, requires more extreme long-term non-pharmaceutical interventions by attempting to reduce the basic reproduction number to less than one. The suppression strategy, which includes stringent population-wide social distancing, home isolation of cases, and household quarantine, was undertaken by communities during the COVID-19 pandemic. Entire cities were placed under lockdown which presented considerable social and economic costs. Because these strategies have limitations and high costs, treatments and immunizations are the needed to respond to pandemic diseases.

New coronavirus disease also known as COVID-19, a severe respiratory acute syndrome in humans is caused by the positive strand RNA SARS-CoV-2 virus which belongs to the beta-coronavirus (β-CoVs) family which has emerged as a severe epidemic, and currently claiming around five million lives and almost 240 million clinical cases worldwide between December 2019 and October 2021. The CoVs family is a class of enveloped, positive-sense single-stranded RNA viruses having an extensive range of natural roots. These viruses can cause respiratory, enteric, hepatic, and neurologic diseases.

The SARS-CoV-2 is composed by a 30 kb genome coding for four structural proteins (SPs) named S, E, M and N, sixteen non-structural proteins (NSPs) as well as some accessory proteins (APs) by ten open reading frames (ORFs). SARS-CoV-2 genome organization is known as 5′-leader-UTR-replicase-S(Spike)-E(Envelope)-M(Membrane)-N(Nucleocapsid)-3′-UTR-poly(A) tail. Accessory genes are interspersed within the structural genes at the 3′-end of the genome. The first Wuhan (people's republic of China) SARS-CoV-2 virus isolated was reported and stored under the National centre of biotechnology information (NCBI), U.S. National Library of Medicine, as the reference sequence NC_045512.2 code. The first genomic virus sequence isolates from worldwide pandemic countries such as Brazil (EPI_ISL_412964 and EPI_ISL_412964), Italy (MT066156.1) and Colombia (EPI_ISL_417924) among many other data from 180 countries were also sequenced and reported. Some population genetic evaluations of more than 100 SARS-CoV-2 genome sequences, shown that these viruses have two major lineages known as S and L, being the L lineage more prevalent than the S lineage.

The virus particle has a diameter of 60˜100 nm and appears round or oval, some representative SARS-Cov-2 models for its structure and genome can be observed in FIG. 1. The virus possesses 10 open reading frames codifying for all structural, non-structural, and accessory proteins as described in FIG. 1.

Among structural proteins a capsid containing the positive single strand (ss) viral RNA anchor a trimer protein named spike (S) coded by the ORF2, is composed by two sub-units Si and Si which harbour a receptor binding domain (RBD) form residues 333 to 527 and binds its human receptor protein the angiotensin converting enzyme 2 (ACE2) which is ubiquitously present in almost all human cells and tissues including lungs, hearth, liver and kidneys organs. Spike possess a so named ectodomain composed by sites 1 and 2 responsible for receptor binding and cell entry having a Kd of ˜15 nM. Later a highly conserved cryptic epitope on the spike RBD was identified. Interestingly, SARS-CoV-2 cell entry through ACE2 was inhibited by specific compounds as reported.

The S protein of SARS-CoV genetic structure can be described as belonging to a transmembrane glycoprotein with a predicted size of 1,255 amino-acids containing a leader sequence from residues 1 to 14, an ectodomain from residues 15 to 1190, a transmembrane domain from residues 1191 to 1227, and a short intracellular tail from residues 1227 to 1255 as elsewhere described. A trimeric spike structure conformation in both close and open forms was recently published.

Similarly, an envelope E gene coded by the ORF4 for a pentamer protein on the virus capsid surface and is coded by the ORF4 and identified as YP_009724392.1. Expression E gene product is a short polypeptide known as a non-glycosylated small transmembrane protein of approximately 10 kDa seem acting as a molecular engine promoting the SARS-CoV-2 assembling in the host cytosolic compartments such as Golgi complex and the endoplasmic reticulum.

A matrix protein product coded by the ORF5 M gene is identified as YP_009724393.1, is a glycoprotein of 25-30 kDa and is highly abundant on the virus surface. It is known to interact with E protein and seem to be relevant for the SARS-CoV-2 maturation and it seems to be a key piece for the virus assembling.

On the other hand, a nucleo-capsid protein is coded by the N gene ORFS of 45-50 kDa, known as being the most conserved among all structure proteins of coronaviruses, it seems being required to virus RNA encapsidation and also seems to be relevant for the virus replication.

The membrane proteins S, E and M are inserted on the intermediate compartment of the virus capsid while the viral RNA undergoes replication and being assembled to the N protein. This RNA-protein complex is associated to the endoplasmic reticulum membrane inserted M protein allowing the virus assembling and migration to the Golgi complex and an eventual virus release from the host cell can occur by exocytosis.

Additionally, 16 non-structural proteins (NSPs) are coded by ORF1a and 1b and actively participate on the virus RNA replication. Some accessory proteins of non-well understood functions are coded by genes from ORF3a, 3b, 6, 7a, 7b, 8, 9b, 9c and 10.

Up to date an important amount of information regarding virus origin, post-infection consequences and immunological trace after different levels of infection among asymptomatic and severe affected patients remains to be obtained since SARS-CoV-2 has just arrived at human being's life and its stablishing and permanence remains also a study matter.

### SUMMARY OF THE INVENTION

The present invention provides amino-acid chains and peptides from SARS-CoV-2 which have been subjected to specific modifications after being strategically screened by a rational selection led to specific virus sequences which have proven to be highly antigenic and immunogenic.

In one embodiment, the present invention provides chemically native and modified peptides have a common a functional motif of formulas Ia and Ib:

₂NH—AA₁—AA₂.AA₃—(AA)ₙ₋₁—(AA)ₙ—CONH₂ Formula Ia

₂NH—AA₁—AA₂.AA₃—(AA)ₙ₋₁—(AA)ₙ—COOH Formula Ib

where AA is any amino-acid, AA₁ is the N-terminus amino-acid residue of a peptide fragment and AAₙ represents the C-terminus residue of peptide chain from 4 to 30 residues included but are not limited to any peptide sequence are either the 20 genetically coded L-amino-acids or their D-enantiomers even those named non-natural amino-acids as well as peptide-bond isostere forms on specific sequence sites. Isostere-bond modification sites are selected from given amino-acid pairs from SARS-CoV-2 selected sequences as single peptide chains herein named as monomer forms.

SARS-CoV-2 peptide selected sequences can be also modified to introduce post-translation elements such as phosphate, methyl, mono and oligo saccharides, small and medium size peptide chains among others. Modifications included but are not limited to Tyr (tyrosine), Gln (glutamine), Asn (asparagine), Ser (serine) and Thr (threonine). Besides, Lys (lysine) residues can be positioned on given peptide motifs to further anchoring other or similar oligo sequences as dendrimers or MAPs (multiple peptide antigen) presenting sequences. Among the realm of peptide modifications, those consisting on introducing solubility inducing motifs can be used among PEG (polyethylene glycol) or even those to be positioned as hydrophobic motifs such as fatty acid small, medium and large size chains including but not limited to palmitic, lauric, stearic, and fumaric acids among all saturated and non-saturated carbon chains.

On the other hand, monomer native and modified SARS-CoV-2 peptide forms are synthesized simultaneously with others having the same amino-acid sequence but including extra amino-acid pairs on both C- and N-terminal peptide ends to serve for obtaining and present them as high molecular weight polymer forms thereof. The formula II represents the group of SARS-CoV-2 peptides synthesized for obtaining a kind of those sequences as polymer forms.

₂NH—Cys—AA₂—AA₃.AA₄—(AA)ₙ₋₁—Cys—CONH₂ Formula IIa

₂NH—Cys—AA₂—AA₃.AA₄—(AA)ₙ₋₁—Cys—COOH Formula IIb

where the N-terminus AA₁ is a Cys (cysteine) residue, as well as the C-terminus (AA)ₙ is a Cys (cysteine) residue. Residues at positions —AA.₂, and —(AA)ₙ₋₁ can be selected from those 20 D- or L-amino-acids such as those side-chain charged polar residues or side-chain functionalized residues such as Gln (glutamine) or Asn (asparagine), or non-polar residues, or non-natural residues, as well as alcohol, carboxylic acids, primary and secondary amino groups or thiol functionalized side-chains or other less complex residues such as Ala (alanine) or Gly (glycine) all considered to be used to introduce spacer motifs that can be located on a given SARS-Cov-2 selected amino-acid sequence. Cathepsins' substrate motifs can be strategically located by flanking those selected SARS-CoV-2 sequences to be efficiently processed by the host immune system.

Cysteine residues located at both C- and N-terminal residues served for obtaining polymer peptides through disulphide-bond oxidation allowed upon being submitted to an oxygen-controlled stream at a pH adjusted between 6.5 to 7.5 under constant stirring at room temperature for a defined time-period from 1 to 24 hours.

All native and modified peptide sequences are aimed to serve as SARS-CoV-2 immuno-detection as well as for COVID-19 prevention tools and can be obtained after performing a rational designing which also included some bioinformatics besides knowledge on immunology, vaccinology, and molecular designing. Compositions and formulations containing these SARS-CoV-2 peptides, methods to obtain their monomers, multimers and polymer forms and uses thereof are also described.

The peptides and compositions thereof described herein, have clinical potential as immuno-detection tools useful for ELISA, immuno-chromatography, and western-blot-based assays as well as, for antibody stimulation in mammals when properly formulated as immunogens on veterinary and human adjuvant and delivery systems and further those would have a potential use for preventing the human COVID-19 disease.

SARS-CoV-2 currently available and reported genome sequences were the basis for obtaining the main amino-acid sequences and peptides of this invention and analyzed as the first step.

A second step consisting in the alignment of most SARS-CoV-2 reported sequence genomes was performed using the Clustal omega tool of EMBL-EBI, Wellcome Genome Campus, Hinxton, Cambridgeshire, CB10 1SD, UK and lead to establishing a ≥96% identity among all compared sequences. Hence, a Basic Local Alignment Search Tool (BLAST) from the NCBI National Center for Biotechnology Information, U.S. National Library of Medicine 8600 Rockville Pike, Bethesda Md., 20894 USA, lead to perform sequence analyses for all ORF coding for structural, non-structural and accessory SARS-CoV-2 proteins

A third sequence analysis step consisting in submitting each ORF coded and translated sequence to identify the presence of LB epitopes, proteasome cleavage sequence sites, HLA-I binding sequences to sensitive and resistant COVID-19 alleles as well as HLA-II binding sequences to susceptible and resistant COVID-19 alleles and phagosome-lysosome protease cleavage sites were performed.

The following remote servers were employed for SARS-CoV-2 sequence analyses: LB epitopes were analyzed with LBtope: linear B-cell Epitope Prediction Server (http://crdd.osdd.net/raghava/lbtope/), ABCpred main page http://crdd.osdd.net/raghava/abcpred/bearing a threshold: 0.51 for a length size from 8 to 16 mere. Bioinformatics tools https://www.iedb.org/, http://tools.iedb.org/main/bcell/, and http://tools.iedb.org/bcell/ were also employed.

Proteasome cleavage servers was http://tools.iedb.org/netchop/help/. HLA-I binding motifs were analyzed with netMHCpan v4.0, as well as the server http://tools.iedb.org/mhci/, was employed.

Selected SARS-CoV-2 infection susceptible HLA-I alleles were HLA-A3.1, HLA-A*25:01, HLA-B*0703, HLA-B67, HLA-B*46:01, HLA-C*0102, HLA-B*5401 and virus resistant alleles HLA-A*0202, HLA-B*1503, HLA-C*1203.

HLA-II binding motifs from SARS-CoV-2 were analyzed with the netMHCpan v3.2 and netMHCpan v2.3 tools of http://tools.iedb.org/mhcii/ and the http://tools.iedb.org/main/bcell/servers were used. Selected SARS-CoV-2 infection susceptible HLA-II alleles were HLA-DRB1*0901, HLA-DQB1*0501, HLA-DQB1*0601 and SARS-CoV-2 infection resistant HLA-II allele was HLA-DRB1*0301.

To identifying phagosome-lysosome substrates the PROSPER server was used having several protease cleavage sites (https://prosper.erc.monash.edu.au/).

For obtaining target sequences to be synthesized some selection criteria were considered. First HLA-II binding peptides showing binding scores from 50 to 100 nM were considered. Those sequences which had simultaneously identified both proteasome cleavage sites, LB epitopes and HLA-I binding sequences were selected. Among those preliminary sequences those matching HLA-II binding sequences, proteasome and phagosome-lysosome cleavage sites were chosen. A map consisting in all preliminary sequences from each SARS-CoV-2 consensus ORFs was built and those coincidences among susceptible and resistant infection HLA-I and HLA-II were eliminated from the list. The non-polymorphic screened unique sequences were considered as the source for a preliminary antigen list to be synthesized by solid phase peptide synthesis using either a t-Boc (tert-butyloxycarbonyl) or a Fmoc (9-methyl fluorenyloxicarbonyl) strategy preferably using the Fmoc chemistry.

Peptides represented by the formula I were proposed from each ORF coding expression products for structural, non-structural, and accessory proteins of SARS-CoV-2.

In a preferred embodiment, the invention provides a combination of structural and conformational modifications on sequences from ORFs 1 to 10, among other target sequences. Monomer peptide sequences are represented by the SVM code followed by an odd number and their polymer peptide forms are represented by the PRE code followed by a pair number. Therefore, a list for peptide sequences from SARS-CoV-2 ORFs 2, 3a, 3b, 4, 5, 7a, 7b and 9 including structural S, E, M and N proteins structural whose designed sequence corresponding to SEQ ID NO:O1 to SEQ ID NO:62, specifically for monomers and for polymer peptide forms are displayed in Table 1 and Table 2.

In another preferred embodiment, the invention provides methods for solid phase peptide synthesis by the Fmoc chemistry, RF-HPLC characterization, mass spectrometry analysis for all obtained peptide antigens derived from the SARS-CoV-2 genome.

The present invention also provides some methods for polymerization of target sequences by either disulfide bridge oxidation or MAPs constructs to obtain high molecular weight forms for each target sequence. In another embodiment the invention describes methods for these polymer peptide formulation in human permitted and veterinary adjuvants and delivery systems to be used for polyclonal and monoclonal stimulations in animal models such as BALB/c mice and other mammals by administration vaccination schemes.

The invention also provides immunochemical methods for assessing recognition of each obtained SARS-CoV-2 sequence by human sera from patients from different COVID-19 infection levels among asymptomatic and those evidencing different levels of infection as well for seroconversion assessment after being vaccine administered with currently permitted vaccines for COVID-19. Methods include but are not limited to ELISA, immuno-chromatography, and immuno-dot base-techniques.

### BRIEF DESCRIPTION OF THE FIGURES

FIG. 1 illustrates the model for the SARS-CoV-2 virus structure.

FIG. 2 shows the human COVID-19 sera reactivity for spike derived designed epitope-peptides.

FIG. 3 features human COVID-19 sera reactivity for spike derived monomer and polymer pooled peptides.

FIG. 4 illustrates human positive COVID-19 sera recognized peptide epitopes from 3a, 3b, E, M, 7a, 7b and N SARS-CoV-2 proteins.

FIG. 5 shows the atigenicity and immunogenicity of Spike-derived designed SARS-CoV-2 peptide sequences.

FIG. 6 describes the antigenicity and immunogenicity of different ORF product-derived designed SARS-CoV-2 peptide sequences.

### DETAILED DESCRIPTION OF THE INVENTION

### SARS-CoV-2 Design and Amino-Acid Sequence Selection

The present invention provides amino-acid chains and peptides from SARS-CoV-2 which are subjected to specific modifications after being screened by a rational selection which lead to obtain highly antigenic and immunogenic peptides.

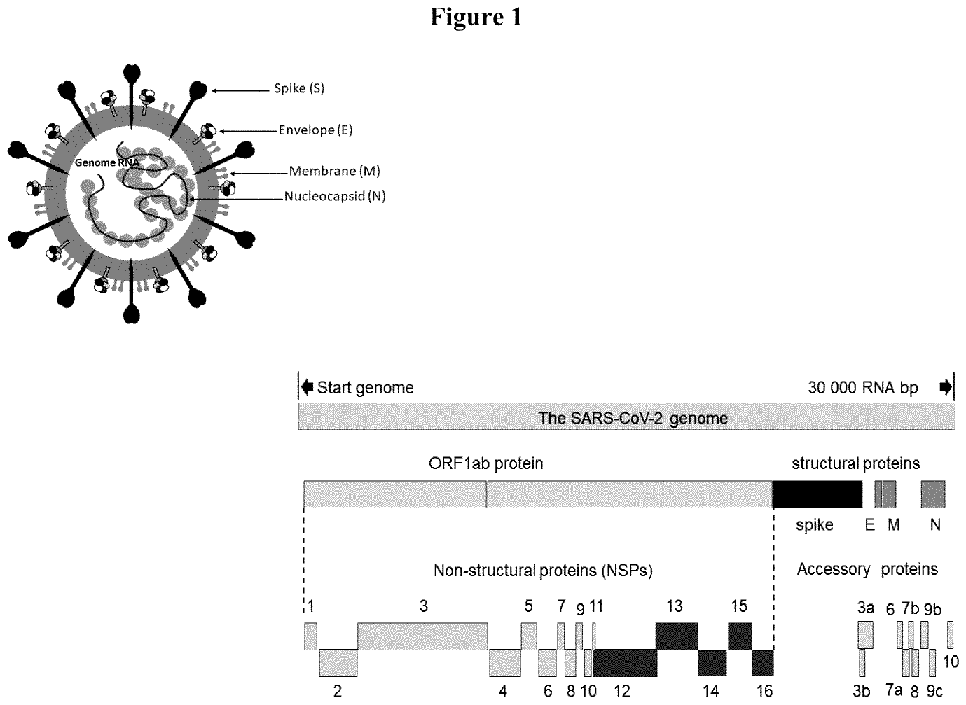

As shown in FIG. 1, a structure model for the SARS-CoV-2 depicts structural S, E, M and N proteins. Structural relationships of the genome and sub-genome mRNAs are shown. ORFs are defined by the published genome sequence. Structural, non-structural, and accessory proteins are displayed including some possible auto-proteolytic processing of ORFs1a and ORFlab polypeptides into protein NSPs1 to 16 are also shown.

In one embodiment, the present invention provides chemically native and modified peptides have a common a functional motif of formulas Ia and Ib:

₂NH—AA₁—AA₂.AA₃—(AA)ₙ₋₁—(AA)ₙ—CONH₂ Formula Ia

₂NH—AA₁—AA₂.AA₃—(AA)ₙ₋₁—(AA)ₙ—COOH Formula Ib

where AA is any amino-acid, AA₁ is the N-terminus amino-acid residue of a peptide fragment and AAₙ represents the C-terminus residue of peptide chain from 4 to 30 residues included but are not limited to any peptide sequence are either the 20 genetically coded L-amino-acids or their D-enantiomers even those named non-natural amino-acids as well as peptide-bond isostere forms on specific sequence sites. Isostere-bond modification sites are selected from given amino-acid pairs from SARS-CoV-2 selected sequences as single peptide chains herein named as monomer forms.

SARS-CoV-2 peptide selected sequences can be also modified to introduce post-translation elements such as phosphate, methyl, mono and oligo saccharides, small and medium size peptide chains among others. Modifications included but are not limited to Tyr (tyrosine), Gln (glutamine), Asn (asparagine), Ser (serine) and Thr (threonine). Besides Lys (lysine) residues can be positioned on given peptide motifs to further anchoring other or similar oligo sequences as dendrimers or MAPs (multiple peptide antigen) presenting sequences. Among the realm of peptide modifications, those consisting on introducing solubility inducing motifs can be used among PEG (polyethylene glycol) or even those to be positioned as hydrophobic motifs such as fatty acid small, medium and large size chains including but not limited to palmitic, lauric, stearic, and fumaric acids among all saturated and non-saturated carbon chains.

On the other hand, monomer native and modified SARS-CoV-2 peptide forms are synthesized simultaneously with others having the same amino-acid sequence but including extra amino-acid pairs on both C- and N-terminal peptide ends to serve for obtaining and present them as high molecular weight polymer forms thereof. The formula II represents the group of SARS-CoV-2 peptides synthesized for obtaining those sequences as polymer forms.

₂NH—Cys—AA₂—AA₃.AA₄—(AA)ₙ₋₁—Cys—CONH₂ Formula IIa

₂NH—Cys—AA₂—AA₃.AA₄—(AA)ₙ₋₁—Cys—COOH Formula IIb

where the N-terminus AA₁ is a Cys (cysteine) residue, as well as the C-terminus (AA)ₙ is a Cys (cysteine) residue. Residues at positions —AA.₂, and —(AA)ₙ₋₁ can be selected from those 20 D- or L-amino-acids such as those side-chain charged polar residues or side-chain functionalized residues such as Gln or Asn, or non-polar residues, or non-natural residues, as well as alcohol, carboxylic acids, primary and secondary amino groups or thiol functionalized side-chains or other less complex residues such as Ala (alanine) or Gly (glycine) all considered to be used to introduce spacer motifs that can be located on a given SARS-Cov-2 selected amino-acid sequence.

Cysteine residues located at both C- and N-terminal residues served for obtaining polymer peptides through disulfide-bond oxidation allowed upon being submitted to an oxygen-controlled stream at a pH adjusted between 6.5 to 7.5 under constant stirring at room temperature for a defined time-period from 1 to 24 hours.

All native and modified peptide sequences are aimed to serve as SARS-CoV-2 immuno-detection as well as for COVID-19 prevention tools and can be obtained after performing a rational designing which also included some bioinformatics besides knowledge on immunology, vaccinology, and molecular designing. Compositions and formulations containing these SARS-CoV-2 peptides, methods to obtain their monomer and polymer forms and uses thereof are also described.

The peptides and compositions thereof described herein, have clinical potential as immuno-detection tools useful for ELISA, immuno-chromatography, and western-blot-based assays as well as, for antibody stimulation in mammals when properly formulated as immunogens on veterinary and human adjuvant and delivery systems and further those have a potential use for preventing the human COVID-19 disease.

Worldwide SARS-CoV-2 reported available genome sequences were the basis for obtaining the main amino-acid sequences and peptides of this invention and analyzed as the first step.

A second step consisting in the alignment of most SARS-CoV-2 reported sequence genomes was performed using the Clustal omega tool of EMBL-EBI, Wellcome Genome Campus, Hinxton, Cambridgeshire, CB10 1SD, UK and lead to establishing a ≥96% identity among all compared sequences. Hence, a Basic Local Alignment Search Tool (BLAST) from the NCBI National Center for Biotechnology Information, U.S. National Library of Medicine 8600 Rockville Pike, Bethesda Md., 20894 USA, lead to perform sequence analyses for all ORF coding for structural, non-structural, and accessory SARS-CoV-2 proteins

A third sequence analysis step consisting in submitting each ORF coded and translated amino-acid sequences to identify the presence of LB epitopes, proteasome cleavage sequence sites, HLA-I binding sequences to sensitive and resistant COVID-19 alleles, as well as HLA-II binding sequences to susceptible and resistant COVID-19 alleles and phagosome-lysosome protease cleavage sites were performed.

The following remote servers were employed for SRAR-CoV-2 sequence analyses: LB epitopes were analyzed with LBtope: linear B-cell Epitope Prediction Server (http://crdd.osdd.net/raghava/lbtope/), ABCpred main page (http://crdd.osdd.net/raghava/abcpred/) bearing a threshold: 0.51 for a length size form 8 to 16 residues. Bioinformatics tools (https://www.iedb.org/, http://tools.iedb.org/main/bcell/), and (http://tools.iedb.org/bcell/) were also employed.

Proteasome cleavage servers was (http://tools.iedb.org/netchop/help/). HLA-I binding motifs were analyzed with netMHCpan v4.0, as well as the server (http://tools.iedb.org/mhci/), was employed.

Selected SARS-CoV-2 infection susceptible HLA-I alleles were HLA-A3.1, HLA-A*2501, HLA-B*0703, HLA-B67, HLA-B*46:01, HLA-C*0102, HLA-B*5401 and virus resistant alleles HLA-A* 0202, HLA-B*1503, HLA-C*1203.

HLA-II binding motifs from SARS-CoV-2 were analyzed with the netMHCpan v3.2 and netMHCpan v2.3 tools of (http://tools.iedb.org/mhcii/) and the (http://tools.iedb.org/main/bcell/) servers were used. Selected SARS-CoV-2 infection susceptible HLA-II alleles were HLA-DRB1*0901, HLA-DQB1*0501, HLA-DQB1*0601 and SARS-CoV-2 infection resistant HLA-II allele was HLA-DRB1*0301.

To identifying phagosome-lysosome substrates the PROSPER server was used having several protease cleavage sites (https://prosper.erc.monash.edu.au/).

For obtaining target sequences to be synthesized some selection criteria were considered. First HLA-II binding peptides showing binding scores from 50 to 100 nM were considered. Those sequences which had simultaneously identified both proteasome cleavage sites, LB epitopes and HLA-I binding sequences were selected. Among those preliminary sequences those matching HLA-II binding sequences, proteasome and phagosome-lysosome cleavage sites were chosen. A map consisting in all preliminary sequences from each SARS-CoV-2 consensus translated ORFs was built and those coincidences among susceptible and resistant infection HLA-I and HLA-II were eliminated from the list. The non-polymorphic screened unique sequences were considered as the source for a preliminary antigen list to be synthesized by solid phase peptide synthesis using either a t-Boc (tert-butyloxycarbonyl) or a Fmoc (9-methyl fluorenyloxicarbonyl) strategy preferably using the Fmoc chemistry.

Peptides represented by the formula I were proposed from each translated ORF coded expression products for structural, non-structural, and accessory proteins of SARS-CoV-2.

In a preferred embodiment, the invention provides a combination of structural and conformational modifications on sequences from ORFs 1 to 10, among other target sequences. Monomer peptide sequences are represented by the SVM code followed by an odd number and their polymer peptide forms are represented by the PRE code followed by a pair number. Therefore, a list for peptide sequences from translated SARS-CoV-2 ORFs 2, 3a, 3b, 4, 5, 7a, 7b and 9 including structural S, E, M and N proteins structural whose designed sequence corresponding to SEQ ID NO:01 to SEQ ID NO:62, specifically for monomers and for polymer peptide forms are displayed in Table 1 and Table 2.

TABLE 1
Single virus monomers (SVM) characteristics

| Consecutive | Working | Amino-acid | | | MW | | e (M⁻¹ | Res | MALDI-TOF |
|---|---|---|---|---|---|---|---|---|---|
| code | code | sequence | Origin | Position | (g/mol) | pI | cm⁻¹) | number | m/z (amu) |
| SEQ ID NO: 01 | SVM-1 | LKPFERDISTEIY | ORF2-S- | 461-483 | 2508.74 | 6.98 | 1280 | 23 | 2509.3 |
| | | QAGSTPSNGV | RBD | | | | | | |
| SEQ ID NO: 03 | SVM-3 | YNSASFSTFKSY | ORF2-S- | 369-392 | 2659.9 | 9.93 | 2560 | 24 | 2660.61 |
| | | GVSPTKLNDLSF | EC1 | | | | | | |
| SEQ ID NO: 05 | SVM-5 | YNYKLPDDFTGS | ORF2-S- | 422-434 | 1630.79 | 6.49 | 2560 | 14 | 1652.35 |
| | | VI | EC2-1 | | | | | | |
| SEQ ID NO: 07 | SVM-7 | NSNNLDSKVGG | ORF2-S- | 437-456 | 2350.54 | 9.78 | 3840 | 20 | 2350.03 |
| | | NYNYLYRLF | EC2-2 | | | | | | |
| SEQ ID NO: 09 | SVM-9 | YQAGSTPSNGVE | ORF2-S- | 473-493 | 2262.39 | 6.56 | 2560 | 21 | 2283.69 |
| | | GFNSYFPLQ | EC3-1 | | | | | | |
| SEQ ID NO: 11 | SVM-11 | FNSYFPLQSYGF | ORF2-S- | 486-506 | 2413.59 | 9.28 | 3840 | 21 | 2435.26 |
| | | QPTNGVGYQ | EC3-2 | | | | | | |
| SEQ ID NO: 13 | SVM-13 | VLSFELLHAPAT | ORF2-S- | 512-532 | 2195.52 | 10.72 | nd | 21 | 2217.46 |
| | | VSGPKKSTN | EC3-3 | | | | | | |
| SEQ ID NO: 15 | SVM-15 | TIDGSSGVVNPV | ORF3A-1 | 248-260 | 1256.41 | 6.33 | 0 | 13 | 1278.87 |
| | | L | | | | | | | |
| SEQ ID NO: 17 | SVM-17 | PVLEPIYDEPTTT | ORF3A-2 | 258-275 | 1971.21 | 3.69 | 1280 | 18 | 1993.41 |
| | | TSVPL | | | | | | | |
| SEQ ID NO: 19 | SVM-19 | VTLAILTALRLS | ORF4-E | 29-47 | 2004.37 | 11.16 | 1280 | 19 | 1998.64 |
| | | AYSSNIV | | | | | | | |
| SEQ ID NO: 21 | SVM-21 | ETNILLNVPLHG | ORFS-M | 115-137 | 2571.97 | 5.36 | 0 | 23 | 2572.01 |
| | | TILTRPLLESE | | | | | | | |
| SEQ ID NO: 23 | SVM-23 | IVFITLSFTLKRK | ORF7A | 107-121 | 1795.17 | 11.58 | 0 | 15 | 1795.71 |
| | | TE | | | | | | | |
| SEQ ID NO: 25 | SVM-25 | SLELQDHNETS | ORF7B | 31-41 | 1271.29 | 4.31 | 0 | 11 | 1271.61 |
| SEQ ID NO: 27 | SVM-27 | KDPNFKDQVILL | ORF9-N-1 | 342-361 | 2398.76 | 9.99 | 1280 | 20 | 2399.32 |
| | | NKHIDAYK | | | | | | | |
| SEQ ID NO: 29 | SVM-29 | KDQVILLNKHID | ORF9-N-2 | 347-366 | 2340.72 | 10.08 | 1280 | 20 | 2340.3 |
| | | AYKTFPPT | | | | | | | |
| SEQ ID NO: 31 | SVM-45 | YQAGSTPSNGV | ORF2-S- | 473-493 | 2261.45 | 10 | 2560 | 21 | 2260.7 |
| | | KGFNSYFPLQ | EC-3-1 | | | | | | |
| SEQ ID NO: 33 | SVM-47 | YQAGSTPSNGV | ORF2-S- | 473-493 | 2247.38 | 9.37 | 2560 | 21 | 2250.54 |
| | | NGFNSYFPLQ | EC-3-1 | | | | | | |
| SEQ ID NO: 35 | SVM-49 | FNSYFPLQSYGF | ORF2-S- | 486-506 | 2462.66 | 9.19 | 5120 | 21 | 2463.2 |
| | | QPTYGVGYQ | EC-3-2 | | | | | | |
| SEQ ID NO: 37 | SVM-51 | ENLLLYIDINGN | ORF1AB: | 1267- | 2410.68 | 4 | 1280 | 22 | 2411.7 |
| | | LHPDSATLVS | NSP3 | 1281 | | | | | |
| SEQ ID NO: 39 | SVM-53 | SDIDITFLKKDAP | ORF1AB: | 1288- | 2108.39 | 4.19 | 1280 | 19 | 2105.43 |
| | | YIVGDV | NSP3 | 1306 | | | | | |
| SEQ ID NO: 41 | SVM-55 | NPTIQKDVLESN | ORF1AB: | 2067- | 2511.82 | 4.44 | 0 | 23 | 2518.98 |
| | | VKTTEVVGDII | NSP3 | 2089 | | | | | |
| SEQ ID NO: 43 | SVM-57 | ILKPANNSLKITE | ORF1AB: | 2089- | 2078.33 | 7.79 | 0 | 19 | 2079 |
| | | EVGHTD | NSP3 | 2107 | | | | | |
| SEQ ID NO: 45 | SVM-59 | SLQNSVLKLKVD | ORF1AB: | 3349- | 3115.67 | 11.25 | 1280 | 27 | 3118.3 |
| | | TANPKTPKYKFVN | SP5 | 3375 | | | | | |
| | | RIQ | | | | | | | |

The abbreviations on Table 1 are:
SEQ for sequences
ORF for open reading frame
MW for molecular weight
pi for isoelectric point
e for molar ellipticity
amu for atomic mass units

TABLE 2
Polymer hybrid elemental (PHE) characteristics

| Consecutive | Working | Amino-acid | | | MW | | e (M−1 | Res | MALDI-TOF |
|---|---|---|---|---|---|---|---|---|---|
| code | code | sequence | Origin | Position | (g/mol) | pI | cm⁻¹) | number | m/z (amu) |
| SEQ ID NO: 02 | PHE-2 | CGLKPFERDISTE | ORF2-S- | 461-483 | 2829.13 | 6.18 | 1280 | 27 | 2832.02 |
| | | IYQAGSTPSNGV | RBD | | | | | | |
| | | GC | | | | | | | |
| SEQ ID NO: 04 | PHE-4 | CGYNSASFSTFK | ORF2-S- | 369-392 | 2980.29 | 9 | 2560 | 28 | 2981.4 |
| | | SYGVSPTKLNDL | EC1 | | | | | | |
| | | SFGC | | | | | | | |
| SEQ ID NO: 06 | PHE-6 | CGYNYKLPDDF | ORF2-S- | 422-434 | 1951.19 | 5.92 | 2560 | 18 | 1952.3 |
| | | TGSVIGC | EC2-1 | | | | | | |
| SEQ ID NO: 08 | PHE-8 | CGNSNNLDSKV | ORF2-S- | 437-456 | 2670.94 | 8.96 | 3840 | 24 | 2673.5 |
| | | GGNYNYLYRLF | EC2-2 | | | | | | |
| | | GC | | | | | | | |
| SEQ ID NO: 10 | PHE-10 | CGYQAGSTPSNG | ORF2-S- | 473-493 | 2582.78 | 5.99 | 2560 | 25 | 2583.53 |
| | | VEGFNSYFPLQG | EC3-1 | | | | | | |
| | | C | | | | | | | |
| SEQ ID NO: 12 | PHE-12 | CGFNSYFPLQSY | ORF2-S- | 486-506 | 2733.99 | 8.11 | 3840 | 25 | 2735.02 |
| | | GFQPTNGVGYQ | EC3-2 | | | | | | |
| | | GC | | | | | | | |
| SEQ ID NO: 14 | PHE-14 | CGVLSFELLHAP | ORF2-S- | 512-532 | 2515.91 | 9.23 | nd | 25 | 2518.24 |
| | | ATVSGPKKSTNG | EC3-3 | | | | | | |
| | | C | | | | | | | |
| SEQ ID NO: 16 | PHE-16 | CGTIDGSSGVVN | ORF3A-1 | 248-260 | 1576.8 | 5.77 | 0 | 17 | 1578.42 |
| | | PVLGC | | | | | | | |
| SEQ ID NO: 18 | PHE-18 | CGPVLEPIYDEPT | ORF3A-2 | 258-275 | 2291.6 | 3.69 | 1280 | 22 | 2294.03 |
| | | TTTSVPLGC | | | | | | | |
| SEQ ID NO: 20 | PHE-20 | CGVTLAILTALR | ORF4-E | 29-47 | 2324.77 | 9.16 | 1280 | 23 | 2325.78 |
| | | LSAYSSNIVGC | | | | | | | |
| SEQ ID NO: 22 | PHE-22 | CGETNILLNVPL | ORFS-M | 115-137 | 2892.36 | 5.36 | 0 | 27 | 2893.41 |
| | | HGTILTRPLLESE | | | | | | | |
| | | GC | | | | | | | |
| SEQ ID NO: 24 | PHE-24 | CGIVFITLSFTLK | ORF7A | 107-121 | 2115.57 | 10.23 | 0 | 19 | 2127.3 |
| | | RKTEGC | | | | | | | |
| SEQ ID NO: 26 | PHE-26 | CGSLELQDHNET | ORF7B | 31-41 | 1591.68 | 4.31 | 0 | 15 | 1593.4 |
| | | SGC | | | | | | | |
| SEQ ID NO: 28 | PHE-28 | CGKDPNFKDQVI | ORF9-N-1 | 342-361 | 2719.15 | 9.04 | 1280 | 24 | 2721.37 |
| | | LLNKHIDAYKGC | | | | | | | |
| SEQ ID NO: 30 | PHE-30 | CGKDQVILLNKH | ORF9-N-2 | 347-366 | 2661.11 | 9.06 | 1280 | 24 | 2662.8 |
| | | IDAYKTFPPTGC | | | | | | | |
| SEQ ID NO: 32 | PHE-46 | CGYQAGSTPSNG | ORF2-S- | 473-493 | 2581.84 | 9.03 | 2560 | 25 | 2583.25 |
| | | VKGFNSYFPLQG | EC-3-1 | | | | | | |
| | | C | | | | | | | |
| SEQ ID NO: 34 | PHE-48 | CGYQAGSTPSNG | ORF2-S- | 473-493 | 2567.77 | 8.12 | 2560 | 25 | 2569.32 |
| | | VNGFNSYFPLQG | EC-3-1 | | | | | | |
| | | C | | | | | | | |
| SEQ ID NO: 36 | PHE-50 | CGFNSYFPLQSY | ORF2-S- | 486-506 | 2783.06 | 8.1 | 5120 | 25 | 2792.45 |
| | | GFQPTYGVGYQ | EC-3-2 | | | | | | |
| | | GC | | | | | | | |
| SEQ ID NO: 38 | PHE-52 | CGENLLLYIDIN | ORF1AB: | 1267- | 2731.07 | 4.16 | 1280 | 26 | 2735.52 |
| | | GNLHPDSATLVS | NSP3 | 1281 | | | | | |
| | | GC | | | | | | | |
| SEQ ID NO: 40 | PHE-54 | CGSDIDITFLKKD | ORF1AB: | 1288- | 2428.78 | 4.19 | 1280 | 23 | 2435.36 |
| | | APYIVGDVGC | NSP3 | 1306 | | | | | |
| SEQ ID NO: 42 | PHE-56 | CGNPTIQKDVLE | ORF1AB: | 2067- | 2832.22 | 4.44 | 0 | 23 | 2835.65 |
| | | SNVKTTEVVGDI | NSP3 | 2089 | | | | | |
| | | IGC | | | | | | | |
| SEQ ID NO: 44 | PHE-58 | CGILKPANNSLKI | ORF1AB: | 2089- | 2398.72 | 6.93 | 0 | 23 | 2400.04 |
| | | TEEVGHTDGC | NSP3 | 2107 | | | | | |
| SEQ ID NO: 46 | PHE-60 | CGSLQNSVLKLK | ORF1AB: | 3349- | 3436.06 | 10.66 | 1280 | 31 | 3440.32 |
| | | VDTANPKTPKY | NSP5 | 3375 | | | | | |
| | | KFVRIQGC | | | | | | | |
| SEQ ID NO: 48 | PHE-62 | CGEIVDTVSALV | ORF1AB: | 5775- | 2793.19 | 8.12 | 1280 | 26 | 2795.68 |
| | | YDNKLKAHKDK | NSP13 | 5796 | | | | | |
| | | SGC | | | | | | | |
| SEQ ID NO: 50 | PHE-64 | CGVSALVYDNK | ORF1AB: | 5780- | 2797.22 | 10.12 | 1280 | 26 | 2802.04 |
| | | LKAHKDKSAQS | NSP13 | 5801 | | | | | |
| | | FKGC | | | | | | | |
| SEQ ID NO: 52 | PHE-66 | GVSLFWNSNVD | ORF1AB: | 6305- | 2457.75 | 8.13 | 6970 | 23 | 2460.54 |
| | | RYPANSIV | NSP14 | 6323 | | | | | |
| SEQ ID NO: 54 | PHE-68 | CGNSIVSRFDTR | ORF1AB: | 6320- | 2421.76 | 9.37 | 0 | 23 | 2424.3 |
| | | VLSNLNLPGGC | NSP14 | 6338 | | | | | |
| SEQ ID NO: 56 | PHE-70 | CGSKSLTENKYS | ORF6 | 47-67 | 2786.01 | 4.01 | 1280 | 25 | 2792.03 |
| | | QLDEEQPLEIDG | | | | | | | |
| | | C | | | | | | | |
| SEQ ID NO: 58 | PHE-72 | CGLTENKYSQLD | ORF6 | 53-66 | 2013.17 | 4.04 | 1280 | 18 | 2015.2 |
| | | EEQPGC | | | | | | | |
| SEQ ID NO: 60 | PHE-74 | CGSLQSSTQHQP | ORF8 | 28-54 | 3542.91 | 7.09 | 9530 | 31 | 3544.15 |
| | | YVVDDPSPIHFY | | | | | | | |
| | | SKWYIGC | | | | | | | |
| SEQ ID NO: 62 | PHE-76 | CGTQHQPYVVD | ORF8 | 33-50 | 2449.68 | 6.04 | 2560 | 22 | 2452.6 |
| | | DPSPIHFYSGC | | | | | | | |

The abbreviations on Table 2 are:
SEQ for sequences
ORF for open reading frame
MW for molecular weight
pi for isoelectric point
e for molar ellipticity
amu for atomic mass units

In another preferred embodiment, the invention provides methods for solid phase peptide synthesis by the Fmoc chemistry, RF-HPLC characterization, mass spectrometry analysis for all obtained peptide antigens derived from the SARS-CoV-2 genome.

The present invention also provides a method for polymerization of target sequences by disulfide bridge formation to obtain high molecular weight forms for each target sequence. In another embodiment, the invention describes methods for these polymer peptide formulation in human permitted and veterinary adjuvants systems to be used for polyclonal and monoclonal stimulations in animal models such as BALB/c mice and other mammals by administration vaccination schemes.

The invention also provides immunochemical methods for assessing recognition of each obtained SARS-CoV-2 sequence by human sera from patients from different COVID-19 infection levels among asymptomatic and those evidencing different levels of infection. Methods included but are not limited to ELISA, immuno-chromatography and immuno-dot assays.

### Methods for Preparing SARS-CoV-2 Selected Peptides

In brief, a procedure for solid-phase peptide synthesis (SPPS) can be followed in organized steps to anchor any peptide chain to an inert resin for a variety of peptide chain lengths as herein used ranging from 4 to more than 30 residues in length. The solid support or inert resin can be a functionalized such as the one called Rink-amide MBHA (methylbenzhydrylamine) resin (100-200 mesh) which led to C-terminus carboxyl amide (—CONH₂) ends. Other resin systems that led to carboxylic acid (—COOH) ends on all the synthesized peptide sequences can be also used. The C-terminal end of a peptide molecule will influence its dipolar moment and thus its solubility and hydrophobic properties. The SPPS steps for including each desired N-alpha protected amino-acid into the peptide chain are performed in successive N-deprotection and activated amino-acid coupling steps and concludes after incorporating the remaining amino-acid sequence as designed, according to the methodologies for SPPS originally developed by Merrifield which was thereafter modified by Houghten for multiple peptide synthesis. In the current invention for simultaneous synthesis of multiple peptide sequences we have employed the Fmoc (9-fluorenylmethyloxycarbonyl) chemistry procedures and used amino-acids are N-alpha-protected with either the Fmoc (9-fluorenylmethyloxycarbonyl) or t-Boc (tert-butyloxycarbonyl) groups. Fmoc chemistry to obtaining all designed SARS-CoV-2-based sequences was chosen for its relatively easy handing and less hazardous management. Details for SARS-CoV-2-based peptide epitopes synthesis are provided in some examples of this invention.

The specific peptides of the invention are described below:

| Consecutive | | Working |
|---|---|---|
| code | Amino-acid sequence | code |
| SEQ ID NO: 01 | LKPFERDISTEIYQAGSTPSNGV | SVM-1 |
| SEQ ID NO: 02 | CGLKPFERDISTEIYQAGSTPSNGVGC | PHE-2 |
| SEQ ID NO: 03 | YNSASFSTFKSYGVSPTKLNDLSF | SVM-3 |
| SEQ ID NO: 04 | CGYNSASFSTFKSYGVSPTKLNDLSFGC | PHE-4 |
| SEQ ID NO: 05 | YNYKLPDDFTGSVI | SVM-5 |
| SEQ ID NO: 06 | CGYNYKLPDDFTGSVIGC | PHE-6 |
| SEQ ID NO: 07 | NSNNLDSKVGGNYNYLYRLF | SVM-7 |
| SEQ ID NO: 08 | CGNSNNLDSKVGGNYNYLYRLFGC | PHE-8 |
| SEQ ID NO: 09 | YQAGSTPSNGVEGFNSYFPLQ | SVM-9 |
| SEQ ID NO: 10 | CGYQAGSTPSNGVEGFNSYFPLQGC | PHE-10 |
| SEQ ID NO: 11 | FNSYFPLQSYGFQPTNGVGYQ | SVM-11 |
| SEQ ID NO: 12 | CGFNSYFPLQSYGFQPTNGVGYQGC | PHE-12 |
| SEQ ID NO: 13 | VLSFELLHAPATVSGPKKSTN | SVM-13 |
| SEQ ID NO: 14 | CGVLSFELLHAPATVSGPKKSTNGC | PHE-14 |
| SEQ ID NO: 15 | TIDGSSGVVNPVL | SVM-15 |
| SEQ ID NO: 16 | CGTIDGSSGVVNPVLGC | PHE-16 |
| SEQ ID NO: 17 | PVLEPIYDEPTTTTSVPL | SVM-17 |
| SEQ ID NO: 18 | CGPVLEPIYDEPTTTTSVPLGC | PHE-18 |
| SEQ ID NO: 19 | VTLAILTALRLSAYSSNIV | SVM-19 |
| SEQ ID NO: 20 | CGVTLAILTALRLSAYSSNIVGC | PHE-20 |
| SEQ ID NO: 21 | ETNILLNVPLHGTILTRPLLESE | SVM-21 |
| SEQ ID NO: 22 | CGETNILLNVPLHGTILTRPLLESEGC | PHE-22 |
| SEQ ID NO: 23 | IVFITLSFTLKRKTE | SVM-23 |
| SEQ ID NO: 24 | CGIVFITLSFTLKRKTEGC | PHE-24 |
| SEQ ID NO: 25 | SLELQDHNETS | SVM-25 |
| SEQ ID NO: 26 | CGSLELQDHNETSGC | PHE-26 |
| SEQ ID NO: 27 | KDPNFKDQVILLNKHIDAYK | SVM-27 |
| SEQ ID NO: 28 | CGKDPNFKDQVILLNKHIDAYKGC | PHE-28 |
| SEQ ID NO: 29 | KDQVILLNKHIDAYKTFPPT | SVM-29 |
| SEQ ID NO: 30 | CGKDQVILLNKHIDAYKTFPPTGC | PHE-30 |
| SEQ ID NO: 31 | YQAGSTPSNGVKGFNSYFPLQ | SVM-45 |
| SEQ ID NO: 32 | CGYQAGSTPSNGVKGFNSYFPLQGC | PHE-46 |
| SEQ ID NO: 33 | YQAGSTPSNGVNGFNSYFPLQ | SVM-47 |
| SEQ ID NO: 34 | CGYQAGSTPSNGVNGFNSYFPLQGC | PHE-48 |
| SEQ ID NO: 35 | FNSYFPLQSYGFQPTYGVGYQ | SVM-49 |
| SEQ ID NO: 36 | CGFNSYFPLQSYGFQPTYGVGYQGC | PHE-50 |
| SEQ ID NO: 37 | ENLLLYIDINGNLHPDSATLVS | SVM-51 |
| SEQ ID NO: 38 | CGENLLLYIDINGNLHPDSATLVSGC | PHE-52 |
| SEQ ID NO: 39 | SDIDITFLKKDAPYIVGDV | SVM-53 |
| SEQ ID NO: 40 | CGSDIDITFLKKDAPYIVGDVGC | PHE-54 |
| SEQ ID NO: 41 | NPTIQKDVLESNVKTTEVVGDII | SVM-55 |
| SEQ ID NO: 42 | CGNPTIQKDVLESNVKTTEVVGDIIGC | PHE-56 |
| SEQ ID NO: 43 | ILKPANNSLKITEEVGHTD | SVM-57 |
| SEQ ID NO: 44 | CGILKPANNSLKITEEVGHTDGC | PHE-58 |
| SEQ ID NO: 45 | SLQNSVLKLKVDTANPKTPKYKFVRIQ | SVM-59 |
| SEQ ID NO: 46 | CGSLQNSVLKLKVDTANPKTPKYKFVR | PHE-60 |
| | IQGC | |
| SEQ ID NO: 47 | EIVDTVSALVYDNKLKAHKDKS | SVM-61 |
| SEQ ID NO: 48 | CGEIVDTVSALVYDNKLKAHKDKSGC | PHE-62 |
| SEQ ID NO: 49 | VSALVYDNKLKAHKDKSAQSFK | SVM-63 |
| SEQ ID NO: 50 | CGVSALVYDNKLKAHKDKSAQSFKGC | PHE-64 |
| SEQ ID NO: 51 | GVSLFWNSNVDRYPANSIV | SVM-65 |
| SEQ ID NO: 52 | GVSLFWNSNVDRYPANSIV | PHE-66 |
| SEQ ID NO: 53 | NSIVSRFDTRVLSNLNLPG | SVM-67 |
| SEQ ID NO: 54 | CGNSIVSRFDTRVLSNLNLPGGC | PHE-68 |
| SEQ ID NO: 55 | SKSLTENKYSQLDEEQPLEID | SVM-69 |
| SEQ ID NO: 56 | CGSKSLTENKYSQLDEEQPLEIDGC | PHE-70 |
| SEQ ID NO: 57 | LTENKYSQLDEEQP | SVM-71 |
| SEQ ID NO: 58 | CGLTENKYSQLDEEQPGC | PHE-72 |
| SEQ ID NO: 59 | SLQSSTQHQPYVVDDPSPIHFYSKWYI | SVM-73 |
| SEQ ID NO: 60 | CGSLQSSTQHQPYVVDDPSPIHFYSKW | PHE-74 |
| | YIGC | |
| SEQ ID NO: 61 | TQHQPYVVDDPSPIHFYS | SVM-75 |
| SEQ ID NO: 62 | CGTQHQPYVVDDPSPIHFYSGC | PHE-76 |

### Compositions Containing SARS-CoV-2 Peptides

The pharmaceutical compositions containing an immuno-therapeutically effective amount or an immuno-stimulating dose of one or more of the SARS-CoV-2 peptide antigens and a carrier and a suitable delivery system. An immuno- stimulating effective amount of a SARS-CoV-2 peptide antigen can be determined according to methods well known in the art. For example, the amount will vary depending on an immunization scheme, subject parameters such as age and size/weight, of an actual or potential presence of a given microorganism or infection, and the administration route.

The present invention relates to compositions comprising one or more SARS-CoV-2 peptide antigens of the invention in an effective immuno-stimulating amount, a pharmaceutically acceptable diluents or adjuvants and delivery system suitable vehicle.

The pharmaceutical composition comprising the SARS-CoV-2 peptide antigens can also contain an antiviral agent. Classes of antivirals that can be used in synergistic therapy with the SARS-CoV-2 peptide antigens of the invention include but are not limited to a wide variety of antiviral agents.

Such pharmaceutical antiviral compositions may be formulated and administered in the form of suitable dosage unit form, as understood in the art for the oral, parenteral (including s.c subcutaneous, i.v intravenous, i.m intramuscular, i.n intranasal and i.p intraperitoneal), topical, vaginal, rectal, dermal, transdermal, intrathoracic, intrapulmonary, and intranasal. In one embodiment, the SARS-CoV-2 peptide antigens of the present invention may comprise from 0.0001% to 50% by weight of such compositions.

To prepare the pharmaceutical composition, the SARS-CoV-2 peptide antigens of the invention are synthesized or obtained otherwise, purified as necessary or desired, and preferably then lyophilized and stabilized. SARS-CoV-2 peptide antigens may then be adjusted to the appropriate immuno-therapeutically concentration and optionally combined with other pharmaceutically acceptable agents.

A pharmaceutical composition comprising a therapeutic peptide in accordance with the present disclosure can be formulated in any pharmaceutically acceptable carrier(s) or excipient(s). As used herein, the term “pharmaceutically acceptable carrier” includes any and all solvents, dispersion media, coatings, antibacterial and antifungal agents, isotonic and absorption delaying agents, and the like that are physiologically compatible. Pharmaceutical compositions can include suitable solid or gel phase carriers or excipients. Exemplary carriers or excipients include calcium carbonate, calcium phosphate, various sugars, starches, cellulose derivatives, gelatin, and polymers such as polyethylene glycols. Exemplary pharmaceutically acceptable carriers include one or more of water, saline, phosphate buffered saline, dextrose, glycerol, ethanol and the like, as well as combinations thereof In many cases it will be preferable to include isotonic agents, for example, sugars, polyalcohols such as mannitol, sorbitol, or sodium chloride in the composition. Pharmaceutically acceptable carriers can further comprise minor amounts of auxiliary substances such as wetting or emulsifying agents, preservatives or buffers, which enhance the shelf life or effectiveness of the therapeutic agents.

The therapeutic peptide can be incorporated into a pharmaceutical composition suitable for parenteral administration. Suitable buffers include but are not limited to, sodium succinate, sodium citrate, sodium phosphate or potassium phosphate. Sodium chloride can be used to modify the toxicity of the solution at a concentration of 0-300 mM (optimally 150 mM for a liquid dosage form). Cryoprotectants can be included for a lyophilized dosage form, principally 0-10% sucrose (optimally 0.5-1.0%). Other suitable cryoprotectants include trehalose and lactose. Bulking agents can be included for a lyophilized dosage form, principally 1-10% mannitol (optimally 2-4%). Stabilizers can be used in both liquid and lyophilized dosage forms, principally 1-50 mM L-Methionine (optimally 5-10 mM). Other suitable bulking agents include glycine, arginine, can be included as 0-0.05%>polysorbate-80 (optimally 0.005-0.01%). Additional surfactants include but are not limited to polysorbate 20 and BRU surfactants.

Therapeutic peptide preparations can be lyophilized and stored as sterile powders, preferably under vacuum, and then reconstituted in bacteriostatic water (containing, for example, benzyl alcohol preservative) or in sterile water prior to injection. Pharmaceutical compositions can be formulated for parenteral administration by injection e.g., by bolus injection or continuous infusion.

Preferably, the polypeptide domains in the therapeutic peptide are derived from the same host in which they are to be administered in order to reduce inflammatory responses against the administered therapeutic agents.

The therapeutic peptide can be administered as a preventive measure (i.e. to avoid infection) at one time or multiple times based on, for example, the half-life of the peptide and the likelihood of one's exposure to a coronavirus. Alternatively, the therapeutic peptide is suitably administered to the patient at one time or over a series of treatments and may be administered to the patient at any time from diagnosis onwards. The therapeutic peptide may be administered as the sole treatment or in conjunction with other drugs or therapies useful in treating the condition in question.

It will be understood that a composition for application, for example by systemic injection, contain an antigenic SARS-CoV-2 peptide in an immuno-stimulating effective amount or an effective amount and dose of a SARS-CoV-2 peptide antigen which may be formulated in adjuvants or delivery systems as a monomer conjugated to another molecule having specificity for a given target cell. The other molecule can be an antibody, ligand, receptor, or other recognition molecule. A standard immunization procedure can be performed by i.e. sub-cutaneous or intra-muscular or even intra-nasal administering an immuno-stimulating amount of either a single or a multiple combined polymer SARS-CoV-2 designed peptides, formulated on an appropriate adjuvant permitted for human use, among Alhydrogel (aluminum hydroxide and phosphates salts) or any other adjuvant human systems such as the saponin QS-21 (Quillaja saponaria), Iscomatrix, Montanide and AS21, monophosphoryl lipid A -MPLA or its modified versions among many other adjuvants systems which activate maturate macrophages, T lymphocytes, B lymphocytes, and NK cells, inflammasomes, pro- and anti-inflammatory signals, inducing inflammatory chemokine and cytokine responses. Delivery systems such as micro- and nano particles and liposomes are also considered in this invention.

Other compositions of the invention comprise one or multiple SARS-CoV-2 peptide antigens of the invention equimolarly combined, an allowed adjuvant and an industrially acceptable vehicle formulated in a delivery system. These compositions may additionally comprise a detergent.

Uses of SARS-CoV-2 Peptides From the Invention

A further object of the present invention is a composition for medical use comprising at least one SARS-CoV-2 peptide as defined above, together with pharmaceutically acceptable diluents and/or vehicles appropriate delivery system and adjuvants. For use in human or veterinary medicine, the composition is preferably in the form of a pharmaceutical dosage form selected from solid, liquid or gels, and combinations thereof, for example, as an eyewash, mouth wash, ointment, aerosol or topical product and injections for parenteral use.

The therapeutic agents in the pharmaceutical compositions can be formulated in a “therapeutically effective amount” or a “prophylactically effective amount”. A “therapeutically effective amount” refers to an amount effective, at dosages and for periods of time necessary, to achieve the desired therapeutic result. A therapeutically effective amount may vary depending on the condition to be treated, the severity and course of the condition, the mode of administration, whether the agent is administered for preventive or therapeutic purposes, the bioavailability of the particular agent(s), the ability of the therapeutic peptide to elicit a desired response in the individual, previous therapy, the age, weight and sex of the patient, the patient's clinical history and response to the agent, the type of the therapeutic peptide used, discretion of the attending physician, etc. A therapeutically effective amount is also one in which any toxic or detrimental effects is outweighed by the therapeutically beneficial effects. A “prophylactically effective amount” refers to an amount effective, at dosages and for periods of time necessary, to achieve the desired prophylactic result.

The following examples illustrate preferred embodiments for carrying-out the invention but do not limit the indicated developments.

### EXAMPLES

### Example 1

### Synthesis of SARS-CoV-2 Peptide Sequences

Selected peptides can be synthesized manually using a solid-phase synthesis by 9-fluorenylmethoxycarbonyl (Fmoc) strategy. Solvents and soluble reagents can be removed by filtration. Washings between deprotection, couplings, and subsequent deprotection steps are carried-out with N,N′-dimethylformamide (DMF) (5×1 min), dichloromethane (DCM) (4×1 min), Isopropyl alcohol (WA) (2×1 min) and DCM (2×1 min) using 1.5 mL of solvent/50 mg of resin for monomer forms and 150 mg for polymer peptides. The Fmoc group is removed from the peptide-resin by two treatments of 15 min with piperidine-DMF (25:75 v/v). Couplings are performed at 20° C. and monitored using standard Kaiser tests for solid-phase peptide synthesis. After Fmoc removal of the commercially available Rink-amide resin (50 mg, 0.46 mmol/g), a first Fmoc-amino-acid (0.115 mmol, 5.0 equiv.) is added with 1-hydroxy benzotriazole (HOBt) (18.2 mg, 0.115 mmol; 5.0 equiv.) and N,N′-dicyclohexylcarbodiimide (DCC) (23.7 mg; 5.0 equiv.) as coupling reagents dissolved in DMF/DCM (7:3, v/v) and the coupling reaction is stirred for 2 hours. Next, the Fmoc group is removed, and a second Fmoc-amino-acid is incorporated to the resin using the same conditions. The Fmoc removal and the coupling reactions of the rest of the Fmoc-amino-acids are carried out under the same conditions using 5 equiv./coupling. Finally, monomer peptide is Fmoc deprotected and cleaved from the resin by treatment with a mixture of trifluoroacetic acid- water-triisopropylsilane (TFA/H₂O/TIS) (95.0:2.5:2.5) for 6 hours followed by filtration and precipitation with cold diethyl ether (Et₂O). Crude products are then triturated 3 times with cold Et₂O, dissolved in the system water-acetonitrile (H₂O:CH₃CN) (9:1 v/v), and then lyophilized. Polymer forms for each synthesized SARS-CoV-2 sequence are cleaved from the Rink-amide resin by treatment with a mixture of trifluoroacetic acid- water- triisopropylsilane-ethane dithiol (TFA/H₂O/TIS/EDT) (94.0:2.5:1.0:2.5) and worked-out as the monomer counter parts for finishing the SPPS procedure. To obtain polymer SARS-CoV.2 based sequences as polymer forms those N- and C-terminal Cys ended sequences are reconstituted to a 4 mg/mL distilled water, then pH is adjusted to 6.5 to 7.5 with a NaOH 3.0 N solution and then submitted to a controlled oxygen stream over a period ranging from 1 to 24 hours as before described. To clean up and eliminate low molecular weight adducts and non-desirable salts a dialysis procedure is then performed to oxidized polymers by employing a 500 Da MWCO Spectrapore® membrane against distilled water changes each hour during a 16-hour time. Then lyophilization under controlled conditions is performed to obtain SARS-CoV-2 sequences as both monomer and polymer forms as a colorless white powder which is subsequently submitted to physicochemical characterization.

### Example 2

### RP-HPLC Characterization of SARS-CoV-2 Peptides

All 30 synthesized molecules were analyzed by reverse phase-high performance liquid chromatography (RP-HPLC) and analyzed by matrix-assisted laser desorption/ionization-time-of-flight (MALDI-TOF) mass spectrometry. HPLC was performed on an Agilent 1200 series chromatograph (Agilent Technologies, Inc., Calif., USA). The system was provided with a Zorbax® HPLC C18 column (4.6×50 mm, 5 μm) (Merck KGaA, Darmstadt, Germany). The system was also provided with a UV-DAD detector adjusted at 220 nm and each component was eluted using a linear acetonitrile gradient a 5% to 95% of B linear gradient during 5 min at a 25° C., with constant flow of 1 mL/min. Mobile phase system was A: H₂O/TFA (99.9:0.1 v/v); and B: CH₃CN/TFA (99.9:0.06 v/v). Each sample was dissolved in a 1:1 mobile phase mixture at a 0.2 mg/200 □L final concentration.

Mass spectrometry characterization was performed on a Bruker Daltonics Microflex II equipment (Billerica, Mass., USA), using the matrix-assisted laser desorption ionization (MALDI) methodology. The time of flight (TOF) technique was used with nitrogen gas laser pulses at 337 nm wavelength, which ensures a minimal number of pulses and greater than 2×10−7 150 μJ/pulse laser energy. Reflection voltage was 20 kV and 20 kV voltage reflector. The matrix used was a-Cyano-4-hydroxycinnamic acid (HCC) (Sigma Chemical Co, St Louis, Mo., USA) 0.1% trifluoroacetic acid (TFA). A volume of 3 μL sample-matrix mixture on a ratio of 0.5 μL sample: 1 μL matrix, were poured to a final concentration of 100 pmol/pL at a laser intensity of 41%. Obtained data can be seen in Table 2.

Each SARS-CoV-2 synthesized sequence was characterized by RP-HPLC analysis and molecular mass was identified by MALDI-TOF, thereby providing homogeneous chromatographic profiles and mass fragmentograms for each molecule, which had a purity higher than 98% whose molecular ions [M +H⁺] reveals ions whose atomic mass units (amu) led to the identification of each molecule. It can also be stated that the synthesis resulted in the obtaining of the expected product in all cases, with purity greater than 95%.

### Example 3

### Human Antibody Detection by Immune-Chemical Tests

Standard Enzyme-linked immunosorbent assays (ELISA) were carried-out for antibody to SARS-CoV-2 detection from human serum samples of COVID-19 infected patients. Thus, polystyrene 96-flat bottom plates (Thermo Fisher Scientific, Waltham, Mass., USA) were overnight immobilized with amounts of each monomer and polymer synthetic antigen in a concentration ranging from 5 μg/mL to 40 μg/mL (carbonates/bicarbonate buffer at a pH of 9.6) at 4° C. Then, three washing cycles of PBS-0.05% Tween-20 and a non-specific binding blocking step with a solution of 1-5% of skimmed-milk in 0.15M (phosphate buffered solution) PBS-0.05% (v/v) tween-20 were performed. Standardized dilutions of human sera samples were served onto flat-bottom 96-wells peptide-ELISA plates and incubated for 1 to 3 hours at 37° C. Therefore, 100 μL of a goat alkaline phosphatase or peroxidase anti human-Ig-conjugate were poured at different dilutions on PBS-Tween-20 and incubated for one extra hour at 37° C. to bind specific human antibodies to SARS-CoV-2 antigen peptides. After carrying-out another cycle of washings, the test was developed by alternatively adding 50 μL of a 1 mg/mL solution of either p-nitrophenylphosphate in 0.1M diethanolamine at a pH 9.8 or a TMB (tetramethyl benzidine)/H₂O₂ substrates to reveal antibodies specific binding to the fixed SARS-CoV-2 viral epitopes by displaying a yellow color which was then recorded on a microplate-reader (Multiskan EX®, Thermo Fisher Scientific, Waltham, Mass., USA) adjusted either at 560 to 570 nm wavelength. Enzyme activity was stopped by adding 50 μL of alternatively 3N NaOH or 1N HCl solution and the microtiter reader system adjusted to 405 or 450 nm wavelength. All samples from COVID-19 patients were collected under Colombian and international ethical regulations (Presidency of the Republic of Colombia, decree number 266, 2006), the world medical association WMA-Declaration of Helsinki of ethical principles for medical research involving human subjects.

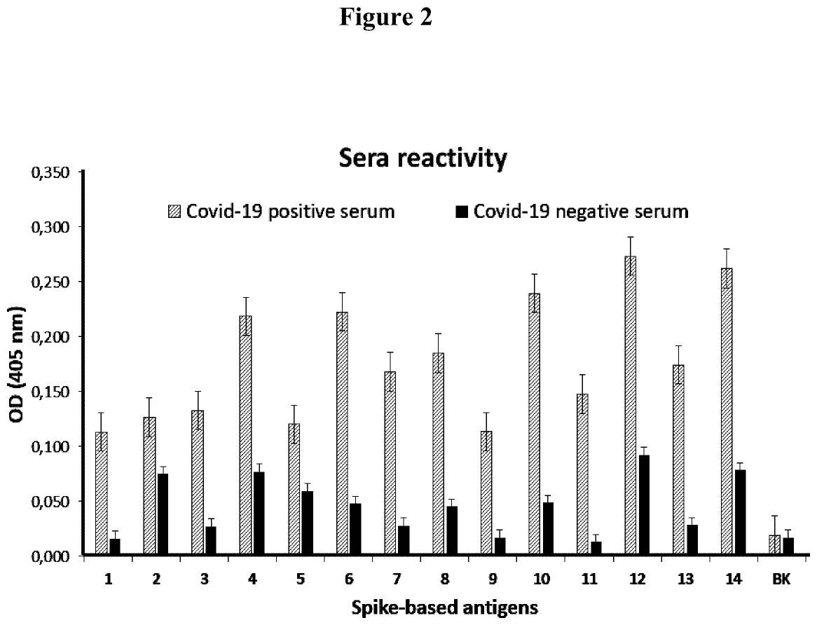

As illustrated in FIG. 2, seven monomeric and their polymer forms were used as ELISA antigens fixed on immunol-1B 96 flat-bottom wells and tested for human COVID-19 patients as well control negative sera. Positive COVID-19 sera specifically and differentially recognized most spike-derived peptide antigens. Odd numbers represent Spike-based epitope monomer forms originally coded as shown in table 1 as SMV as well as, pair numbers represent polymer forms thereof originally coded as PRE followed by pair numbers. Bk on FIG. 2 stands for the blank.

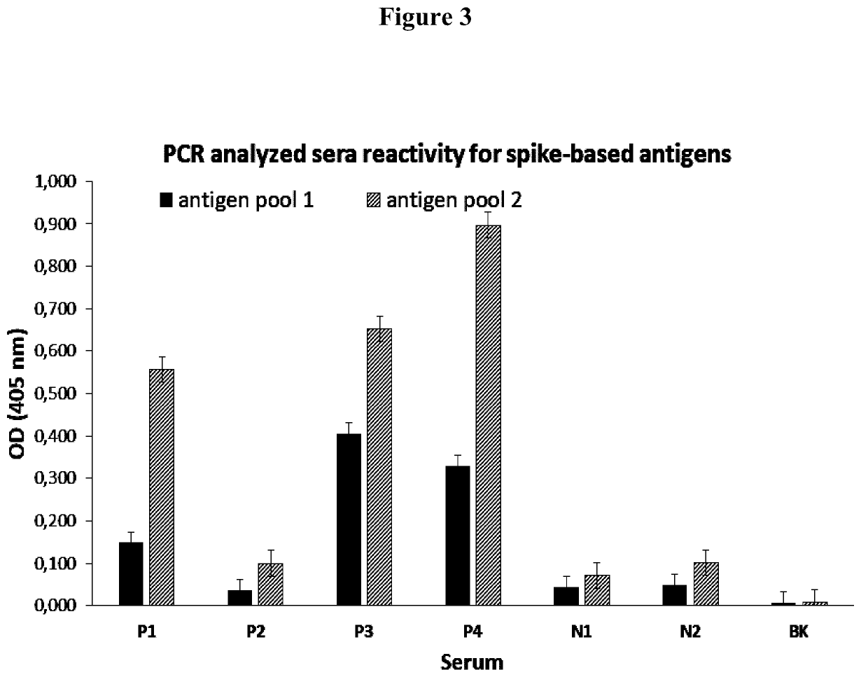

As shown in FIG. 3, seven monomer and their polymer forms from SARS-CoV-2 produced antigens were pooled into two groups and used each as ELISA antigens, which were fixed on immunol-1B polystyrene 96 flat-bottom well plates and tested for human COVID-19 patient's recognition, as well negative COVID-19 diagnosed serum were also used. Positive COVID-19 sera preferentially recognized most spike-derived polymer pooled peptide antigens displaying higher OD values. P for PCR positive diagnosed patients' serum, N for PCR negative patients' serum and Bk for the blank.

### Example 4

### Neutralization Infection Capacity of SARS-CoV-2 Lineages by Antibodies From Mice Immunized With Designed Sequences

To assess the capacity of the herein described prototype vaccine formulations for COVID-19 based on single components or mixtures thereof to generate protection mediated by neutralization, BALB/c mice groups were immunized. Blood samples were obtained after a second boost and then assayed in in vitro tests faced to the SARS-CoV-2 circulation lineages in Colombia (B.1, B.1.111, B.1.111+E48K) infected a human cell line, as well as Vero E6 cells, in the presence and absence of mice antibodies. In a biosafety laboratory equipped with material necessary for the test, which was adapted from the previously reported by Algaissi A., et al—Scientific Reports (2020) 10:16561, Doi:/10.1038/s41598-020-73491-5

Briefly, sero neutralization procedure consisted in seeding 96-well plates with a concentration of 1.5×10⁵ Vero E6 cells/mL, allowing cell adherence between 16 and 24 hours, by incubation at 37° C. with 5% CO₂. In the biological safety hood, in new sterile 96-well plates, dilutions of the previously inactivated sera were performed by adding 60 μL of DMEM to 2% into all wells. Subsequently adding extra 48 μL of DMEM at 2% calf fetal sera CFS, to the wells. Thereafter adding 12 μL of the respective sample, in duplicate in testing rows, thus obtaining a 1:10 dilution. As recommended, different plates should be used when performing the micro sero neutralization test against different virus strains. Followed serial dilutions were performed from one row to another starting from row A to H. Then, 60 μL of diluted virus suspension were added to all wells in agreement to the experimental design, and then 60 μL of DMEM 2% CSF medium to all wells of column 12 were also added. Serum-virus mixtures were then incubated for 1 hour at 37° C. at 5% CO₂. Lastly, 100 μL of the serum-virus dilutions were transferred plate to the plate containing VERO E6 cells, considering the designed experiment. A final incubation at 37° C. at 5% CO₂ was allowed for 3 days. Read cell density on the plates according to the presence of a clear cytopathic effect and the validation of the controls by using a light inverted microscope and the corresponding neutralizing titers for each test serum was so determined.

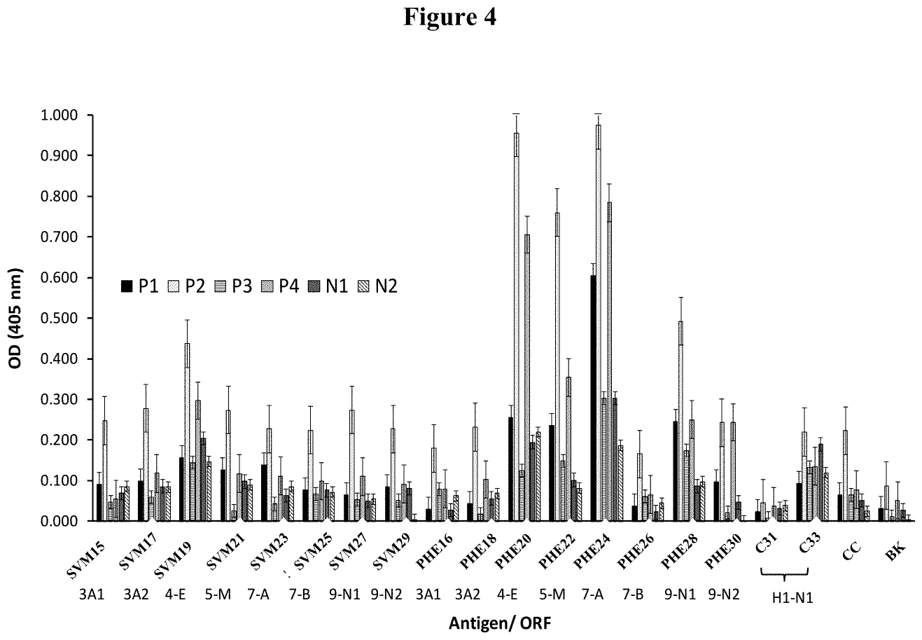

As described in FIG. 4, four COVID-19 positive sera specifically react to individually monomer and polymer peptide antigens designed form SARS-CoV-2 virus used each independently for recognition assays. Peptide-epitope sequences derived from proteins E, M, N and ORFs 3a and 7a and 7b were strongly recognized for most COVID-19 positive diagnosed patients, being these antigens presented as both monomer and polymer forms. Monomer forms are SMV coded followed by an odd number and polymer forms thereof PRE coded followed by a pair number. C31 and C33 are sequences from H1N1 influenza virus used as the controls and CC represents a reaction performed in absence of any serum sample and BK is used for the blank.

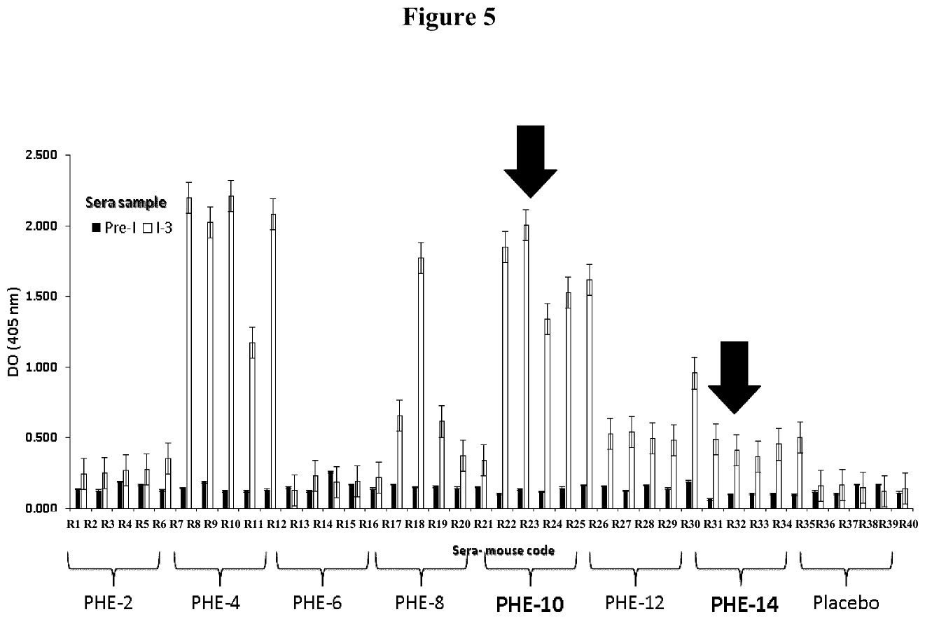

In FIG. 5, there is shown the antigenicity and immunogenicity of Spike-derived designed SARS-CoV-2 peptide sequences. BALB/c mice groups were subcutaneously (s.c.) administered on a scheme of three-dose administration with 100 μL of a formulation of each polymeric peptide on Alhydrogel (3%). Sera neutralizing antibodies were detected in mice immunized with PRE-10 and PRE-14 systems derived from spike as denoted by bold arrows.

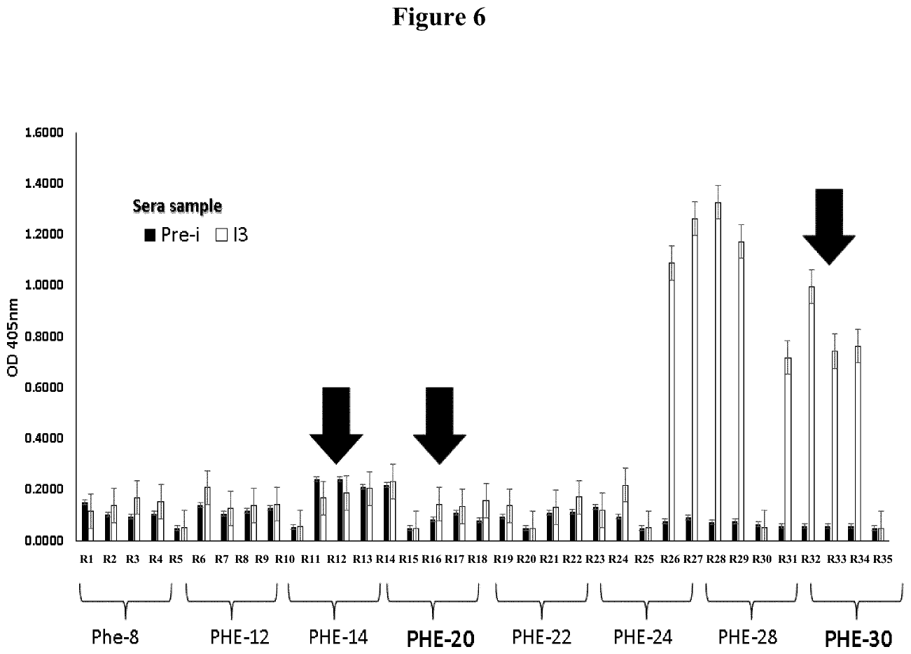

FIG. 6 further illustrates the antigenicity and immunogenicity of different ORF product-derived designed SARS-CoV-2 peptide sequences. BALB/c mice groups were subcutaneously s.c. administered on a scheme of three-dose administration with 100 μL of a formulation of each polymeric peptide on Alhydrogel (3%). Sera neutralizing antibodies were detected in mice immunized with PRE-20 (envelope-derived epitope) and PRE-30 (nucleocapsid-derived epitope) systems derived from spike as denoted by bold arrows.

All literature and similar materials cited in this application including, but not limited to, patents, patent applications, articles, books, treatises, and internet web pages, regardless of the format of such literature and similar materials, are expressly incorporated by reference in their entirety for any purpose as if they were entirely denoted. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents. In the event that one or more of the incorporated literature and similar materials defines or uses a term in such a way that it contradicts that term's definition in this application, this application controls.

Although the foregoing description contains many specifics, these should not be construed as limiting the scope of the present invention, but merely as providing illustrations of some of the presently preferred embodiments. Similarly, other embodiments may be devised without departing from the spirit or scope of the present invention. Features from different embodiments may be employed in combination. The scope of the invention is, therefore, indicated and limited only by the appended claims and their legal equivalents rather than by the foregoing description. All additions, deletions and modifications to the invention as disclosed herein which fall within the meaning and scope of the claims are to be embraced thereby.

### REFERENCES

1. https://www.hopkinsmedicine.org/coronavirus/index.html accessed october 25th, 2020.

2. Yan R., Zhang Y., Li Y, Xia L., Guo Y., Zhou Q., Brogden N. K., Brogden K. A. Structural basis for the recognition of the SARS-CoV-2 by full-length human ACE2. Science 10.1126/science.abb2762 (2020).

3. Wrapp D., Wang N., Corbett K. S., Goldsmith J. A., Hsieh C.-L., Abiona O. Graham B. S., McLellan J. S. Cryo-EM structure of the 2019-nCoV spike in the prefusion conformation. Science eabb2507 (2020). doi:10.1126/science.abb2507 Medline

4. Meng Y., Wu N. C., Zhu X, Lee C. C-D, So R. T. Y., Lv H,. Mok C. K, Wilson I. A. A highly conserved cryptic epitope in the receptor-binding domains of SARS-CoV-2 and SARS-CoV. Science. 10.1126/science.abb7269 (2020).

5. Hoffmann M., Kleine-Weber H., Schroeder S., Kruger N., Herrler T., Erichsen S., Schiergens T. S., Herrler G., Wu N-H., Nitsche A., Muller M. A. Drosten C., Pohlmann S. SARS-CoV-2 Cell Entry Depends on ACE2 and TMPRSS2 and Is Blocked by a Clinically Proven Protease Inhibitor. Cell 181, 1-10. (2020).

6. Huo J, Zhao Y, Ren J, Zhou D, Duyvesteyn H M E, Ginn H M, Carrique L, Malinauskas T, Ruza R R, Shah P N M, Tan T K, Rijal P, Coombes N, Bewley K R, Tree J A, Radecke J, Paterson N G, Supasa P, Mongkolsapaya J, Screaton G R, Carroll M, Townsend A, Fry E E, Owens R J, Stuart D I. Neutralization of SARS-CoV-2 by Destruction of the Prefusion Spike. Cell Host Microbe. 2020 Sep. 9;28(3):445-454.e6. doi: 10.1016/j.chom.2020.06.010. Epub 2020 Jun. 19. Erratum in: Cell Host Microbe. 2020 Sep. 9;28(3):497. PMID: 32585135; PMCID: PMC7303615.

7. Jurtz V., Paul S., Andreatta M., Marcatili P., Peters B., Nielsen M. NetMHCpan-4.0: Improved Peptide—MHC Class I Interaction Predictions Integrating Eluted Ligand and Peptide Binding Affinity Data. The Journal of Immunology. 2017.

8. O'Donnell T. J., Rubinsteyn A., Bonsack M. Riemer A. B., Laserson U., Hammerbacher. MHCflurry: Open-Source Class I MHC Binding Affinity Prediction. J. Cell Syst 2018.7(1):129-132.e4. doi: 10.1016/j.cels.2018.05.014. 2018.

9. Wang S. F., Chen K. H., Chen M., W Y Li., Chen Y. J., et al. Human-Leukocyte Antigen Class I Cw 1502 and Class II DR 0301 Genotypes Are Associated with Resistance to Severe Acute Respiratory Syndrome (SARS) Infection. Viral Immunol. 24, (5).2011.

10. Merrifield R. B. Solid phase synthesis. The synthesis of a tetrapeptide. J. Am. Chem. Soc., 85:2149 (1963).

11. Houghten R. A. General method for the rapid solid-phase synthesis of large numbers of peptides: Specificity of antigen-antibody interaction at the level of individual amino acids. Proc. Natl. Acad. Sci. Vol. 82, p. 5131-5135 (1985).

12. Carpino L.; Han G. The 9-Fluorenylmethoxycarbonyl Amino-Protecting Group. J. Org. Chem. 1979, 44, 3739-3739.

13. Hansen R. A. and Oddo A., Fmoc Solid-Phase Peptide Synthesis, chapter 5 in Gunnar Houen (ed.), Peptide Antibodies: Methods and Protocols, Methods in Molecular Biology, vol. 1348, DOI 10.1007/978-1-4939-2999-3_5, © Springer Science+Business Media New York 2015

14. Behrendt R., White P. and Offer J. Advances in Fmoc solid-phase peptide synthesis. J. Pept. Sci. 2016; 22: 4-27. DOI 10.1002/psc.2836.

15. Bashiri S., Koirala P., Toth I. and Skwarczynski M. Carbohydrate Immune Adjuvants in Subunit Vaccines. Pharmaceutics 2020, 12, 965; doi:10.3390/pharmaceutics12100965.

16. Lozano J. M., Espejo F., Diaz D., Salazar L. M., Rodriguez J., Pinzon C., et al. Reduced amide pseudopeptide analogues of a malaria peptide possess secondary structural elements responsible for induction of functional antibodies which react with native proteins expressed in Plasmodium falciparum erythrocyte stages. J. Pept Res. 1998;52(6):457-69.

What is claimed is:

1. A set of specific monomer SARS-CoV-2 derived and rationally designed antigenic peptides comprising formulas Ia and Ib:
₂NH—AA₁—A₂.AA₃—(AA)ₙ₋₁—(AA)ₙ—CONH₂ Formula Ia
₂NH—AA₁—AA₂.AA₃—(AA)ₙ₋₁—(AA)ₙ—COOH Formula Ib
wherein AA is any amino-acid, AA₁ is the N-terminus amino-acid residue of a peptide fragment and AAₙ represents the C-terminus residue of a peptide chain having from 4 to 30 residues including but not limited to any peptide sequence that are either the 20 genetically coded L-amino-acids or their D- enantiomers also including non-natural amino-acids, as well as peptide-bond isosteric forms or peptide-bond surrogates on specific sequence sites.

2. The antigenic peptide of claim 1, wherein the isostere-bond modification sites are selected from given amino-acid pairs from SARS-CoV-2 selected sequences as single peptide chains and as single viral monomer forms so-coded SVM.

3. Native and modified polymer hybrids of SARS-CoV-2 peptides having the same amino-acid sequence of their monomers but including extra amino-acid pairs on both C- and N-terminal peptide ends comprising the formulas IIa and IIb:
₂NH—Cys—AA₂—AA₃.AA₄—(AA)ₙ₋₁—Cys—CONH₂ Formula IIa
₂NH—Cys—AA₂—AA₃.AA₄—(AA)ₙ₋₁—Cys—COOH Formula IIb
wherein the N-terminus Amino Acid is a Cys (cysteine) residue, the C-terminus Amino Acid is a Cys (cysteine) residue and the residues at positions —AA₂, AA₃, AA₄ and —(AA)ₙ₋₁ are selected from those genetically coded 20 L- or their enantiomers D-amino-acids including but not limited to those having side-chain charged polar residues or side-chain functionalized residues such as Gln or Asn, or non-polar residues, or non-natural residues, as well as alcohol, carboxylic acids, primary and secondary amino groups or thiol functionalized side-chains or residues as Ala (alanine) or Gly (glycine) wherein said residues allow for introduction of spacer motifs that can be located on a given SARS-Cov-2 selected amino-acid sequence.

4. The Native and modified polymer hybrids of SARS-CoV-2 peptides of claim 3, wherein the Cysteine residues located at both C- and N-terminal residues are used for obtaining polymer peptides through disulfide-bond oxidation.

5. The Native and modified polymer hybrids of claim 4, wherein said disulfide-bond oxidation is conducted using a controlled oxygen stream at a pH adjusted between 6.5 to 7.5 under constant stirring at room temperature for a time-period from about 1 to about 24 hours.

6. The Native and modified polymer hybrids of claim 3, selected from the group consisting of multimeric systems selected from the group conssiting of Cys-linear polymers, dendrimers and multi antigen presenting system -MAPs.

7. A method for the systematic screening of SARS-CoV-2 epitope sequences for selecting and designing immunogenic and antigenic probes, said method characterized by the use of sequence homology and identity comparison of genomic and hypothetical translated gene products, affinity for COVID-19 sensitive and resistant HLA-I and II classes alleles, HLA-II differential selection, L-B epitope content, proteasomal cleavage sites, as well as substrates for elastases, cathepsins among other phagosome-lysosome proteolytic entities; and sequence refinement with remote server bioinformatics tools for a rational design that lead to synthesis and testing of those most representative SARS-CoV-2 sequences.

8. Peptide sequences derived from SARS-CoV-2 ORFs 1ab, 2, 3a, 3b, 4, 5, 6, 7a, 7b, 8 and 9, selected from the group consisting of SEQ ID NO:01, SEQ ID NO: 02, SEQ ID NO: 03, SEQ ID NO:04, SEQ ID NO:05, SEQ ID NO:06, SEQ ID NO:07, SEQ ID NO:08, SEQ ID NO:09, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31 SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61 and SEQ ID NO:62.

9. Pharmaceutical compositions and formulations for vaccination comprising at least one of the peptides of claim 1, and wherein said compositions include pharmaceutically acceptable carriers, diluents or delivery systems, and veterinary and human acceptable adjuvants.

10. Pharmaceutical compositions and formulations for vaccination comprising at least one of the peptides of claim 3, and wherein said compositions include pharmaceutically acceptable carriers, diluents or delivery systems, and veterinary and human acceptable adjuvants.

11. Pharmaceutical compositions and formulations for vaccination comprising at least one of the peptides of claim 8, and wherein said compositions include pharmaceutically acceptable carriers, diluents or delivery systems, and veterinary and human acceptable adjuvants.

12. The composition of claim 9, characterized in that it is a pharmaceutical dosage form selected from the group consisting of solids, liquids, gels, and combinations thereof.

13. The compositions of claim 8, characterized by further comprising one or more of all L- or D-amino-acids or non-natural amino-acids, or isostere peptide-bonds on selected peptide sites or posttranslational modifications thereof including methyl, phosphate, mono and oligo saccharides, fatty acids groups on specific SARS-CoV.2 designed peptide antigens.

14. The compositions of claim 9, characterized by further comprising one or more of all L- or D-amino-acids or non-natural amino-acids, or isostere peptide-bonds on selected peptide sites or posttranslational modifications thereof including methyl, phosphate, mono and oligo saccharides, fatty acids groups on specific SARS-CoV.2 designed peptide antigens.

15. A method for using any of the amino-acid sequences presented in claim 8, as antibody detection tools for the COVID-19 disease and seroconversion of vaccinated populations. Detection methods included but are not limited to ELISA, immunoblot, immuno-chromatography and western blot like procedures.

16. A method of treating a subject for the prevention of an infection caused by the SARS-CoV-2 virus, wherein said method comprises the step of administering an immuno-therapeutically effective amount of a peptide composition of of claim 8.

17. A method of treating a subject for the prevention of an infection caused by the SARS-CoV-2 virus, wherein said method comprises the step of administering an immuno-therapeutically effective amount of a peptide composition of of claim 9.

18. A method for stimulating and producing neutralizing poly- and monoclonal antibodies, directed to those epitopes described in claim 1, by administering doses of a formulation of those selected components or mixtures thereof, by different administration pathways under immunization dose-schemes on higher vertebrates and different species such as BALB/c mice, rabbits, monkeys among others.

19. A method for stimulating and producing neutralizing poly- and monoclonal antibodies, directed to those epitopes described in claim 1, by administering doses of a formulation of those selected components or mixtures thereof, by different administration pathways under immunization dose-schemes on higher vertebrates and different species such as BALB/c mice, rabbits, monkeys among others.

20. A method for stimulating and producing neutralizing poly- and monoclonal antibodies, directed to those epitopes described in claim 8, by administering doses of a formulation of those selected components or mixtures thereof, by different administration pathways under immunization dose-schemes on higher vertebrates and different species such as BALB/c mice, rabbits, monkeys among others.
